(19) United States
(12) Patent Application Publication
(10) Pub. No.: US 2026/0069212 A1
(43) Pub. Date: Mar. 12, 2026

(54) Portable Biomechanical Assessment System

(71) Applicant: LUBU Technologies, Inc., West Hollywood, CA (US)

(72) Inventors: Luca BURATTO, Milan (IT); Ahmed ISSAOUI, Marina del Rey, CA (US)

(21) Appl. No.: 19/375,221

(22) Filed: Oct. 31, 2025

Related U.S. Application Data
(63) Continuation-in-part of application No. 19/325,375, filed on Sep. 10, 2025.
(60) Provisional application No. 63/692,760, filed on Sep. 10, 2024.

(51) Int. Cl. A61B 5/00; A61B 5/01; A61B 5/103; A61B 5/11

(52) U.S. Cl. CPC A61B 5/6812; A61B 5/0002; A61B 5/01; A61B 5/1038; A61B 5/112; A61B 2562/0219; A61B 2562/0247; A61B 2562/0271; A61B 2562/222

(57) ABSTRACT

The various embodiments described herein include a portable biomechanical assessment system that includes a detachable electronic device having first connector pins, a frame configured to secure the detachable electronic device, a transitional layer that includes a cut-out configured to accommodate the frame and the detachable electronic device, a base layer that at least partially surrounds the transitional layer, and a printed circuit board disposed inside the cut-out of the transitional layer. The base layer includes a cavity that is configured to accommodate the transitional layer, the printed circuit board includes second connector pins, and the frame is configured to secure the detachable electronic device in position so that electrical contact is maintained between the first connector pins of the detachable electronic device and the second connector pins of the printed circuit board while the detachable electronic device is attached in the frame. A foot orthotic is also disclosed herein.

### RELATED APPLICATIONS

This application is a continuation-in-part of U.S. application Ser. No. 19/325,375, filed Sep. 10, 2025, titled “Portable Biomechanical Assessment System,” which claims the benefit of and priority to U.S. Provisional Patent Application No. 63/692,760, filed Sep. 10, 2024, entitled “Intelligent Wearable System,” each of which is incorporated by reference herein in its entirety.

### TECHNICAL FIELD

The disclosed embodiments relate generally to wearable technology and more specifically to a portable biomechanical assessment system for implementing health and performance monitoring.

### BACKGROUND

The field of wearable technology, especially in the context of health and fitness, has seen significant advancements in recent years. Traditional wearables, such as wristbands and smartwatches, have been focused primarily on tracking superficial metrics like step count, heart rate, and calories burned. However, these devices often lack the capability to provide in-depth biomechanical analysis. Footwear-based systems have been introduced, but these typically offer limited or incomplete technology such as requiring external electronics or lacking sensors and/or live data transmission capabilities. Furthermore, existing analysis tools are predominantly used in clinical settings or sports laboratories and involve bulky equipment that is not suitable for continuous, everyday wear. Thus, existing technologies are not able to provide a comfortable and wearable system that is capable of capturing complete data of a user's movement.

### SUMMARY

Disclosed are new portable biomechanical assessment systems that are include a comfortable foot orthotic that can capture information regarding a user's movement.

The portable biomechanical assessment systems disclosed herein leverage high-sensitivity sensors and capture detailed and comprehensive gait and biomechanical information. The portable biomechanical assessment system includes integrated electronics for real-time information capture. Further, the portable biomechanical assessment system is designed with user comfort in mind, allowing a user to leverage the power of movement sensing without compromising comfort or support. Existing solutions for wearable technology lack the fully integrated hardware and electronics and user-friendly design that is provided by the systems disclosed herein.

In accordance with some embodiments, a foot orthotic includes (i) a device frame configured to secure a detachable electronic device and (ii) a composite core. The composite core includes a transitional layer that includes a cut-out configured to accommodate the device frame and the detachable electronic device. The composite core includes a base layer that at least partially surrounds the transitional layer and includes a cavity that is configured to accommodate the transitional layer.

In accordance with some embodiments, a portable biomechanical assessment system includes: (i) a detachable electronic device having one or more first connector pins, (ii) a device frame configured to secure the detachable electronic device, (iii) a transitional layer that includes a cut-out configured to accommodate the device frame and the detachable electronic device, (iv) a base layer that at least partially surrounds the transitional layer, and (v) a printed circuit board disposed inside the cut-out of the transitional layer. The base layer includes a cavity that is configured to accommodate the transitional layer. The printed circuit board includes one or more second connector pins. The device frame is configured to secure the detachable electronic device in position so that electrical contact is maintained between the one or more first connector pins of the detachable electronic device and the one or more second connector pins of the printed circuit board while the detachable electronic device is attached in the device frame.

In various circumstances, the portable biomechanical assessment system of the present disclosure has the following advantages over conventional wearable systems. First, in accordance with some embodiments, the portable biomechanical assessment system of the present disclosure includes integrated sensors and electronics for sensitive and accurate real-time monitoring of a user's movement. Second, the portable biomechanical assessment system includes a user friendly detachable electronic device that can be easily swapped between different shoes (or foot orthotics) without requiring additional setup. Third, the portable biomechanical assessment system of the present disclosure includes a uniquely designed foot orthotic that prioritizes user comfort. The foot orthotic allows the presence of the sensors and electronics to be sensorially ‘invisible’ to the user during use, thereby providing the user with needed comfort and support along with added sensing capabilities. Fourth, the portable biomechanical assessment system of the present disclosure is capable of real-time or near real-time data transmission, allowing for real-time or near-real time monitoring of the user's movement.

Thus, methods and systems are disclosed for a portable biomechanical assessment system that includes detachable electronics paired with a foot orthotic with integrated sensors for health and performance monitoring. Such systems may complement or replace conventional methods and systems of wearable technology.

### BRIEF DESCRIPTION OF THE DRAWINGS

For a better understanding of the aforementioned systems, as well as additional systems that provide portable biomechanical assessment system for health and performance monitoring, reference should be made to the Description of Embodiments below, in conjunction with the following drawings in which like reference numerals refer to corresponding parts throughout the figures.

FIG. 1A is a bottom perspective view of a portable biomechanical assessment system in accordance with some embodiments.

FIG. 1B is a top view of the portable biomechanical assessment system shown in FIG. 1A in accordance with some embodiments.

FIG. 1C is another top perspective view of the portable biomechanical assessment system shown in FIG. 1A in accordance with some embodiments.

FIG. 1D is a lateral side view of the portable biomechanical assessment system shown in FIG. 1A in accordance with some embodiments.

FIG. 1E is a medial side view of the portable biomechanical assessment system shown in FIG. 1A in accordance with some embodiments.

FIG. 1F is a bottom view of the portable biomechanical assessment system shown in FIG. 1A in accordance with some embodiments.

FIG. 1G is an exploded cross-sectional view of the portable biomechanical assessment system shown in FIG. 1B in accordance with some embodiments.

FIG. 1H is an exploded view of the portable biomechanical assessment system shown in FIG. 1B in accordance with some embodiments.

FIG. 2A is a bottom perspective view of the portable biomechanical assessment system shown in FIG. 1A with the detachable electronic device removed, in accordance with some embodiments.

FIG. 2B is a top view of the printed circuit board shown in FIG. 2A, in accordance with some embodiments.

FIG. 2C is a bottom view of the printed circuit board shown in FIG. 2A, in accordance with some embodiments.

FIG. 2D is a side view of the printed circuit board shown in FIG. 2A, in accordance with some embodiments.

FIG. 2E is a back view of the printed circuit board shown in FIG. 2A, in accordance with some embodiments.

FIG. 2F is a front view of the printed circuit board shown in FIG. 2A, in accordance with some embodiments.

FIGS. 3A-3G show a detachable electronic device of the portable biomechanical assessment system shown in FIG. 1A, in accordance with some embodiments.

FIGS. 4A-4C show a device frame of the portable biomechanical assessment system shown in FIG. 1A, in accordance with some embodiments.

FIGS. 4D-4H show an alternative embodiment of a device frame, in accordance with some embodiments.

FIG. 5A is a top perspective view of the device frame and the transitional layer of the portable biomechanical assessment system shown in FIG. 1A, in accordance with some embodiments.

FIG. 5B is a bottom perspective view of the device frame and the transitional layer of the portable biomechanical assessment system shown in FIG. 1A, in accordance with some embodiments.

FIG. 5C is a top view of the device frame and the transitional layer of the portable biomechanical assessment system shown in FIG. 1A, in accordance with some embodiments.

FIG. 5D is a bottom view of the device frame and the transitional layer of the portable biomechanical assessment system shown in FIG. 1A, in accordance with some embodiments.

FIGS. 5E and 5F are side views of the device frame and the transitional layer of the portable biomechanical assessment system shown in FIG. 1A, in accordance with some embodiments.

FIG. 6 shows a base layer of the portable biomechanical assessment system shown in FIG. 1A in accordance with some embodiments.

FIGS. 7A-7D are cross-sectional views of a portable biomechanical assessment system in accordance with some embodiments.

FIGS. 8A and 8B show sensors and cables of the portable biomechanical assessment system shown in FIG. 1A in accordance with some embodiments.

FIGS. 9A-9C show how to insert the detachable electronic device into the device frame in accordance with some embodiments.

FIG. 9D shows how to remove the detachable electronic device from the device frame in accordance with some embodiments.

Reference will now be made to embodiments, examples of which are illustrated in the accompanying drawings. In the following description, numerous specific details are set forth in order to provide a thorough understanding of the present invention. However, it will be apparent to one of ordinary skill in the art that the present invention may be practiced without requiring these specific details.

### DESCRIPTION OF EMBODIMENTS

A portable biomechanical assessment system of the present disclosure provides a wearable laboratory for biomechanical movement assessment. The portable biomechanical assessment system includes a foot orthotic (e.g., an insole, also referred to as a foot orthotic) that is coupled to a detachable electronic device. The foot orthotic includes pressure sensors, and the detachable electronic device includes an inertial measurement unit, both of which capture information regarding the wearer's movements. The foot orthotic has a thin profile that is supportive and sensorially conceals the detachable electronic device from the wearer's underfoot, making the portable biomechanical assessment system a unique system that provides support akin to conventional foot orthotics with the added ability for movement monitoring and assessment. The thin profile also makes the portable biomechanical assessment system suitable for use in athletic shoes that have a slimmer profile, such as cleats. Additionally, the portable biomechanical assessment system can easily be swapped between shoes, allowing the wearer maximum freedom in leveraging the abilities of the portable biomechanical assessment system both during athletic activities and leisurely activities (each of which may require different footwear). The foot orthotic also includes embedded metadata, allowing for automatic and ad hoc connection with the detachable electronic devices without the need for reconfiguration by the user.

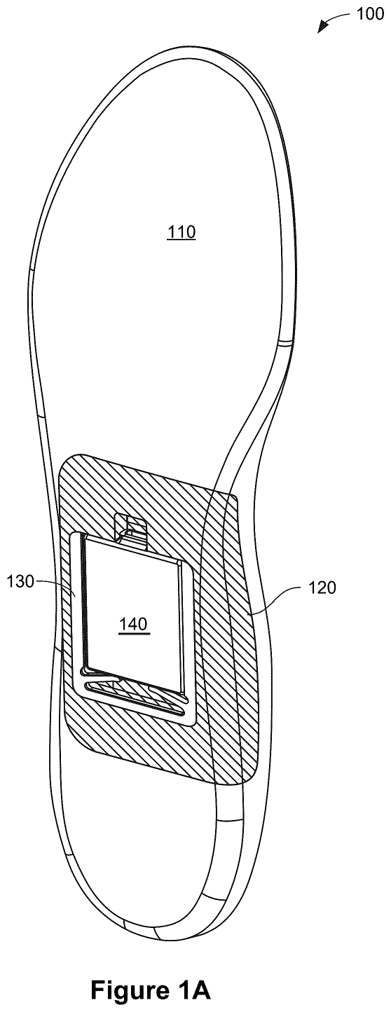

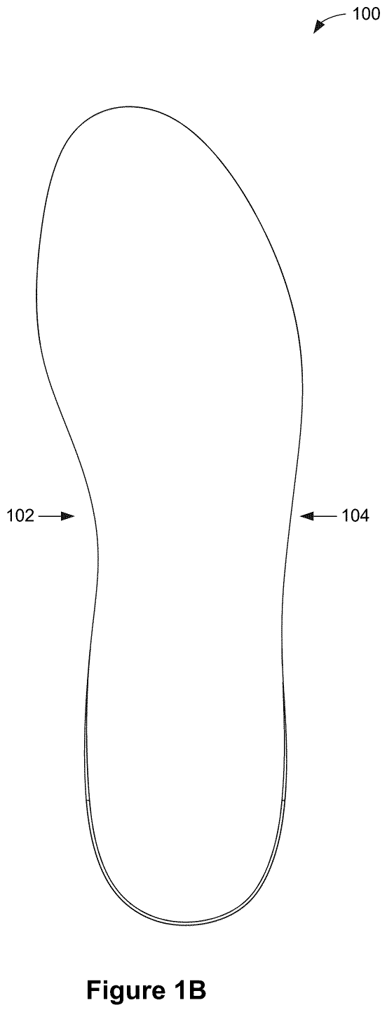

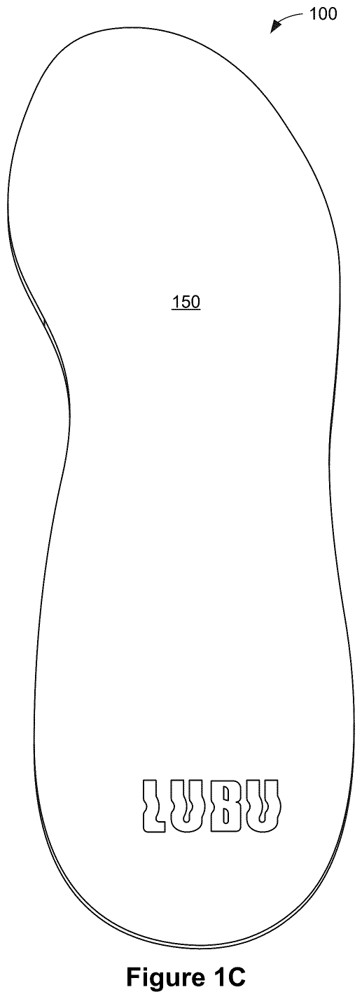

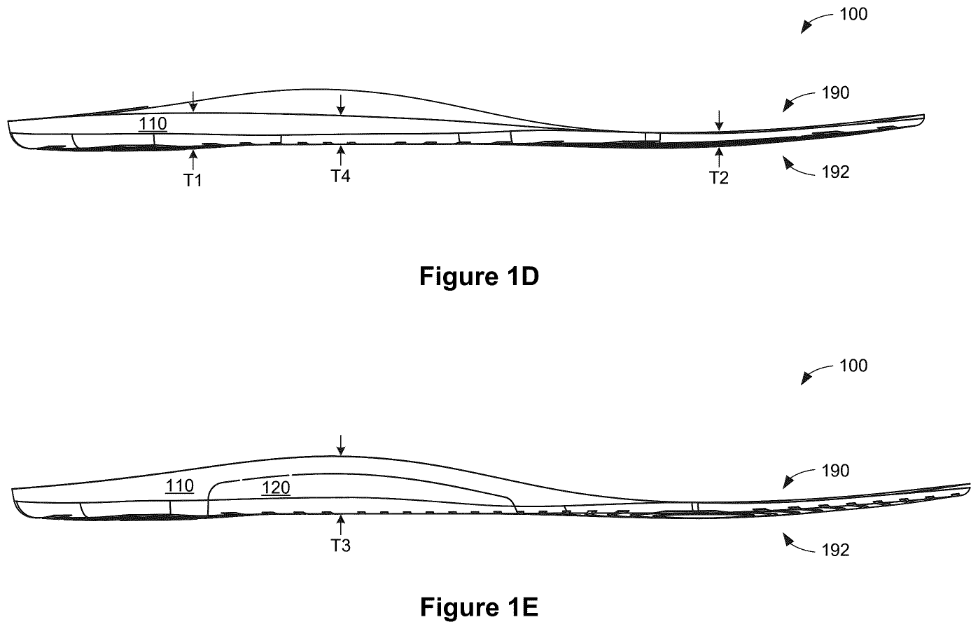

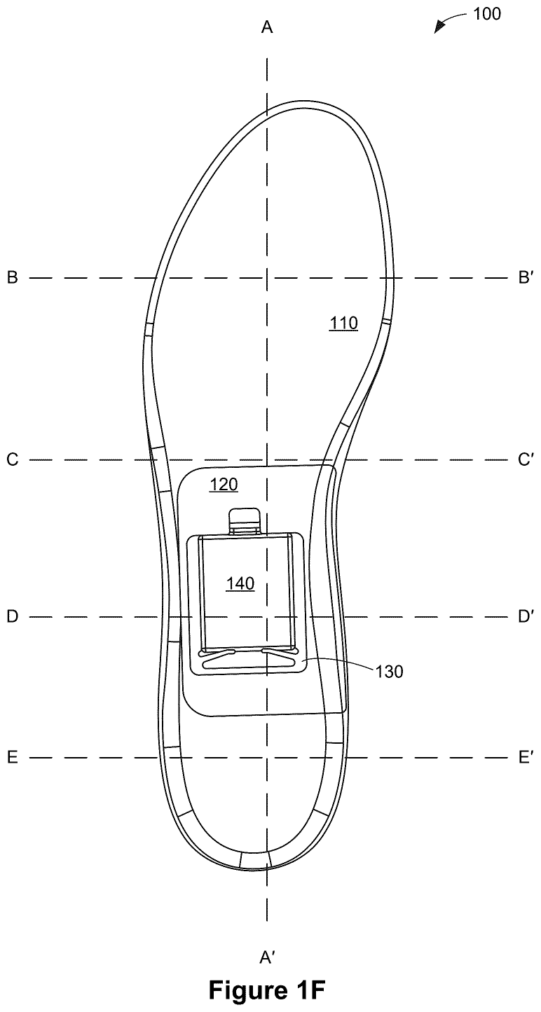

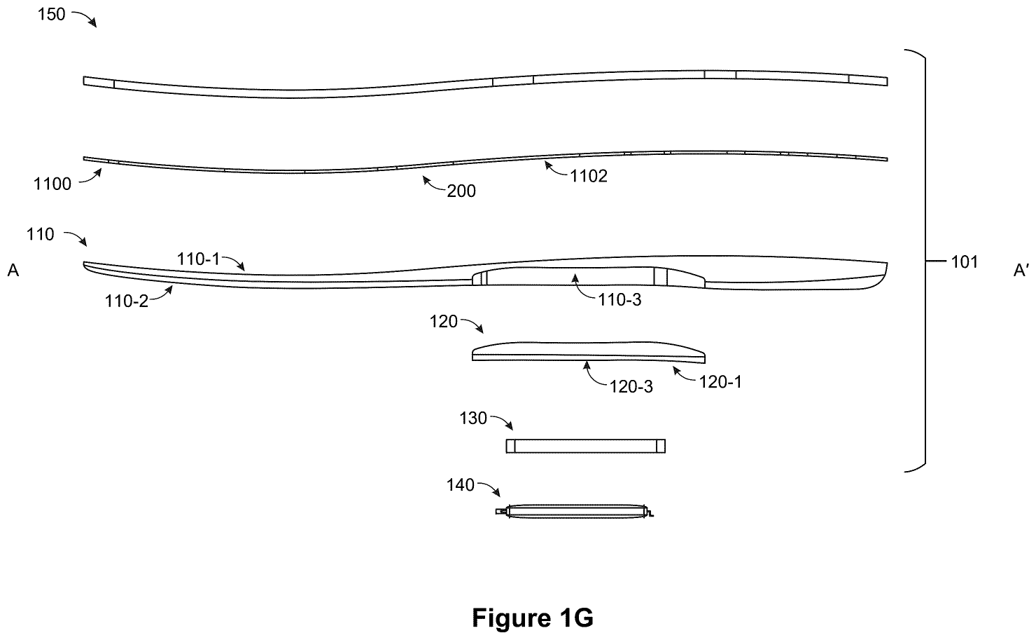

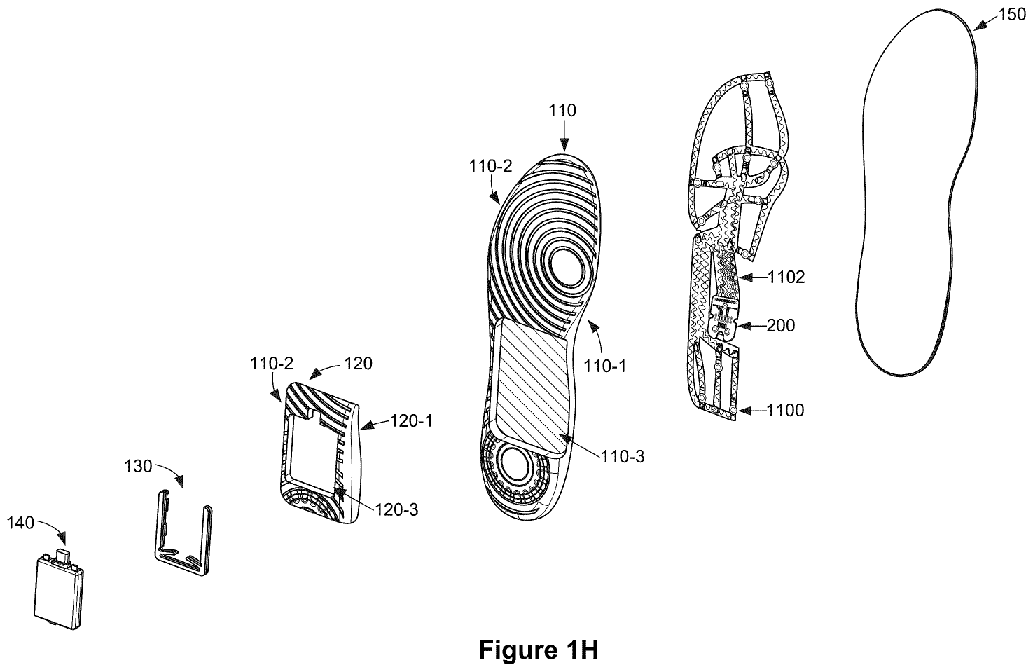

FIGS. 1A-1H provide various views of a portable biomechanical assessment system 100 in accordance with some embodiments.

FIG. 1A is a bottom perspective view of the portable biomechanical assessment system 100 in accordance with some embodiments. The portable biomechanical assessment system 100 includes a composite core 101 that includes a base layer 110, a transitional layer 120 (filled with a diagonal pattern), and a device frame 130. Together, the base layer 110, the transitional layer 120, and the device frame 130 are referred to as the composite core 101. As shown, the portable biomechanical assessment system 100 is a foot orthotic (e.g., insole) that is configured for wearing inside of a shoe. The portable biomechanical assessment system 100 also includes a detachable electronic device 140, which is configured to be easily removed. The device frame 130 is configured to secure the detachable electronic device 140, when it is inserted into the device frame 130.

The portable biomechanical assessment system 100 can be worn inside of a shoe with the detachable electronic device 140 inserted (e.g., attached) or with the detachable electronic device 140 removed (e.g., not inserted, or detached). The composite core 101 is configured to provide support (e.g., foot support, sole support, arch support, heel support, and/or fore foot support) to a wearer (e.g., user) of the portable biomechanical assessment system 100. It provides support both when the detachable electronic device 140 is inserted and when the detachable electronic device 140 is not inserted.

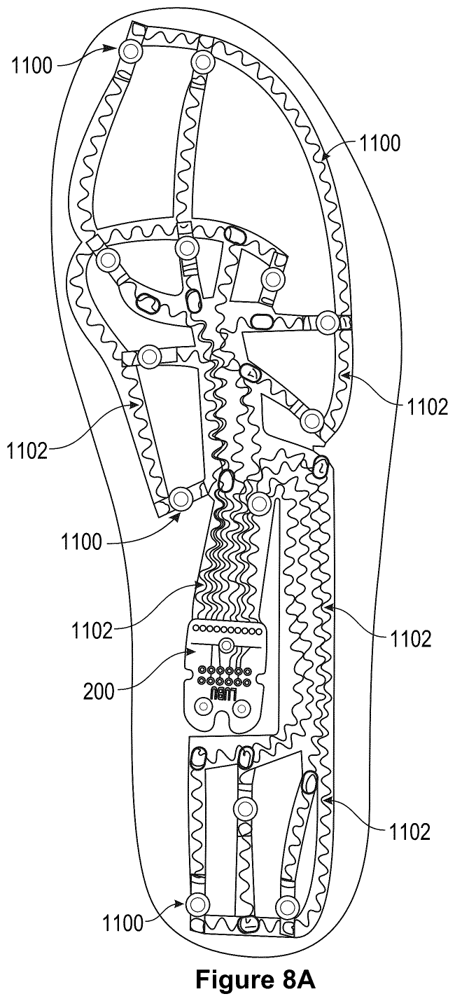

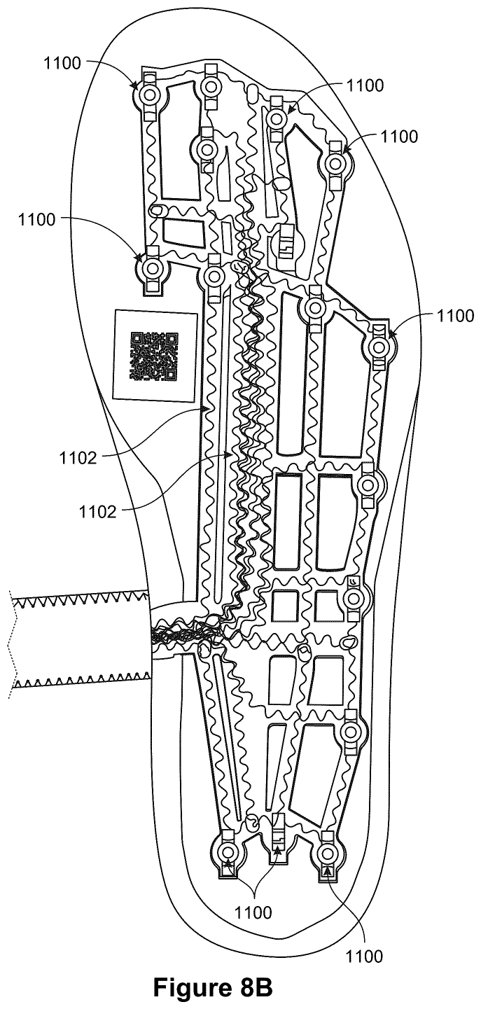

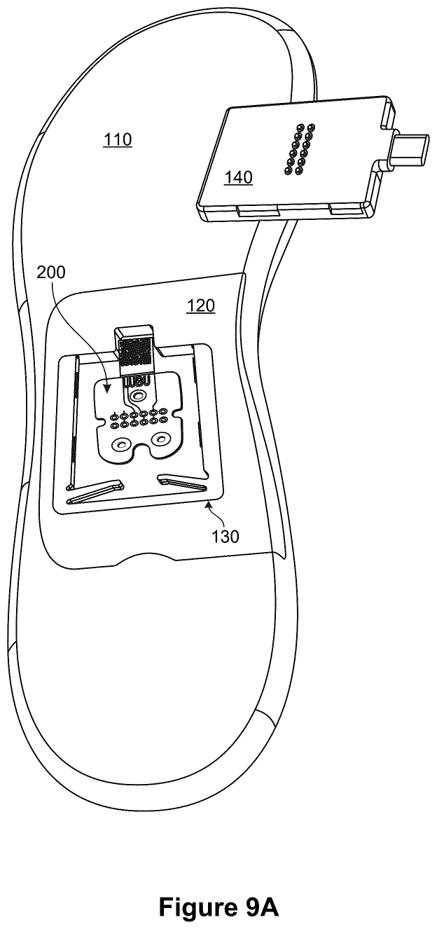

In some embodiments, the portable biomechanical assessment system 100 also includes one or more sensors. Details regarding the sensors are provided below with respect to FIGS. 8A and 8B.

In some embodiments, the portable biomechanical assessment system 100 also includes a cover layer, described below with respect to FIG. 1C.

FIG. 1B is a top view of the portable biomechanical assessment system 100 shown in FIG. 1A in accordance with some embodiments. FIG. 1B shows the medial side 102 and the lateral side 104 of the portable biomechanical assessment system 100. The medial side 102 refers to the portion of the portable biomechanical assessment system 100 that is configured to be positioned near the midline (e.g., relative to the lateral side 104) of the wearer when the portable biomechanical assessment system 100 is worn (e.g., as part of a shoe). For example, the hallux (e.g., the big toe) and the medial longitudinal arch of the wearer's foot are located toward the medial side 102 when wearing the portable biomechanical assessment system 100. The lateral side 104 refers to the portion of the portable biomechanical assessment system 100 that is configured to be positioned further away from the midline (e.g., relative to the medial side 102) of the wearer when the portable biomechanical assessment system 100 is worn (e.g., as part of a shoe). For example, the pinky toe and the lateral longitudinal arch of the wearer's foot are located toward the lateral side 104 when wearing the portable biomechanical assessment system 100.

In some embodiments, the portable biomechanical assessment system 100 also includes a cover layer 150 that is disposed on top of the base layer 110. The cover layer 150 can be clearly seen FIG. 1C, which is a top perspective view of the portable biomechanical assessment system 100. The cover layer 150 is configured to be adjacent to the sole of a wearer's foot when the portable biomechanical assessment system 100 is worn (e.g., as part of a shoe). In some embodiments, the cover layer 150 is composed of a breathable material, such as an antibacterial mesh. In some embodiments, the cover layer 150 is configured to provide comfort to the wearer by wicking away sweat from the bottom of the wearer's foot and preventing slipping of the wearer's foot. In some embodiments, the cover layer 150 is configured to protect the surfaces and materials (such as a surface of the base layer 110 and/or sensors embedded as part of the composite core 101) of the portable biomechanical assessment system 100.

FIG. 1D is a lateral side view of the portable biomechanical assessment system 100 shown in FIG. 1A in accordance with some embodiments. In this view, the transitional layer 120, the device frame 130, and the detachable electronic device 140 are not visible.

FIG. 1E is a medial side view of the portable biomechanical assessment system 100 shown in FIG. 1A in accordance with some embodiments. In this view, the device frame 130 and the detachable electronic device 140 are not visible.

The profile, shape, and thicknesses of the portable biomechanical assessment system 100 are primarily (e.g., largely or for the most part) determined by the profile, shape, and thicknesses of the composite core 101 (which includes the base layer 110, transitional layer 120, and device frame 130). The composite core 101 includes a top surface 190 that is configured to be adjacent to a sole of a wearer while the portable biomechanical assessment system 100 is being used (e.g., being worn as part of a shoe). The composite core 101 also includes a bottom surface 192 that is opposite the top surface 190. The composite core 101 also includes a perimeter (e.g., the outer perimeter, the edge, or the outer edge) which is at least partially raised so that the top surface 190 includes concave contours and the bottom surface 192 includes convex contours.

In some embodiments, a portion of the edge (e.g., the outer edge, the perimeter, or the outer perimeter) of the portable biomechanical assessment system 100 corresponding to a medial hindfoot portion of the portable biomechanical assessment system 100 has a height T1 (e.g., thickness, edge height, or edge thickness) that is no greater than any of: 10.5 mm, 10.6 mm, 10.7 mm, 10.8 mm, 10.8 mm, 10.9 mm, 11 mm, 11.1 mm, 11.2 mm, 11.3 mm, 11. mm, 11.5 mm, 12.0 mm, 12.5 mm, and 13.0 mm.

In some embodiments, a forefoot portion of the portable biomechanical assessment system 100 has a thickness T2 that is 4.5 mm, 4.8 mm, 5 mm, 5.4 mm, 6.0 mm, or 7 mm.

In some embodiments, a portion of the edge of the portable biomechanical assessment system 100 (e.g., a portion of the edge of the composite core 101) corresponding to a medial midfoot portion (e.g., corresponding to the arch of a foot) of the portable biomechanical assessment system 100 has a height T3 (e.g., thickness, edge height, or edge thickness) that larger than a height T4 (e.g., thickness, edge height, or edge thickness) of a portion of the edge of the portable biomechanical assessment system 100 corresponding to the lateral midfoot portion. In some embodiments, the height T4 is 15.0 mm, 16.5 mm, 18 mm, or 20.0 mm.

In some embodiments, such as when the portable biomechanical assessment system 100 is configured to be worn in athletic shoes and/or low-profile shoes such as sports cleats, the midfoot region of the portable biomechanical assessment system 100 (e.g., of the composite core 101) has thickness (e.g., any of T3 and T4) that is no greater than 5.5 mm, no greater than 6.0 mm, no greater than 6.5 mm, or no greater than 7.0 mm.

FIG. 1F is a bottom view of the portable biomechanical assessment system 100 shown in FIG. 1A in accordance with some embodiments. FIG. 1F includes design details (e.g., ridges and lines) on the bottom of the portable biomechanical assessment system 100 that are not shown in FIG. 1A.

FIG. 1G is an exploded cross-sectional view of the portable biomechanical assessment system shown in FIG. 1B, in accordance with some embodiments. FIG. 1H is an exploded view of the portable biomechanical assessment system 100.

The base layer 110 includes a first surface 110-1 (e.g., the top surface) and a second surface 110-2 (e.g., the bottom surface), which is opposite to the first surface 110-1. The first surface 110-1 is configured to be positioned adjacent to the sole of a wearer's foot when the portable biomechanical assessment system 100 is worn. The second surface 110-2 (e.g., the bottom surface) is configured to be positioned adjacent to the midsole of a shoe (or adjacent to the ground) when the portable biomechanical assessment system 100 is worn or inserted into a shoe. When the portable biomechanical assessment system 100 is worn, the first surface 110-1 of the base layer 110 is located between the sole of the wearer's foot and the second surface 110-2. The base layer 110 also includes a cavity 110-3 disposed on the second surface 110-2 of the base layer 110. The cavity 110-3 is configured to accommodate the transitional layer 120 so that the base layer 110 at least partially surrounds the transitional layer 120.

The transitional layer 120 includes a first surface 120-1 and a second surface 120-2 that is opposite to the first surface 120-1. The transitional layer 120 is disposed inside the cavity 110-3 of the second surface 110-2 of the base layer 110 so that the first surface 120-1 of the transitional layer 120 is adjacent to and in contact with the portion of the second surface 110-2 of the base layer 110 corresponding to the cavity 110-3. The second surface 120-2 of the transitional layer 120 includes a cut-out 120-3 (e.g., a hole) that is configured to accommodate the device frame 130 and the detachable electronic device 140. The device frame 130 is disposed inside the cut-out 120-3. The detachable electronic device 140, when inserted, is held in position (e.g., in place, secured) by the device frame 130 so that the detachable electronic device 140 is also disposed inside the cutout 120-3. As shown in FIGS. 1D, 1E, and 1G, when the detachable electronic device 140 is inserted into the device frame 130, the second surface 110-2 of the base layer 110, the second surface 120-2 of the transitional layer 120, a surface of the device frame 130, and a surface of the detachable electronic device 140 form a bottom surface of the foot orthotic (e.g., insole) that is the portable biomechanical assessment system 100.

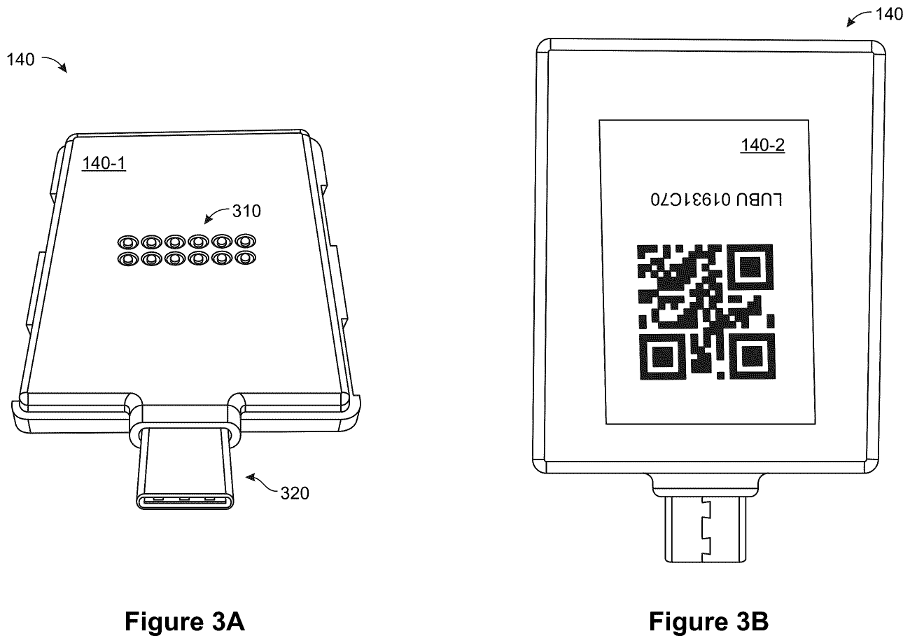

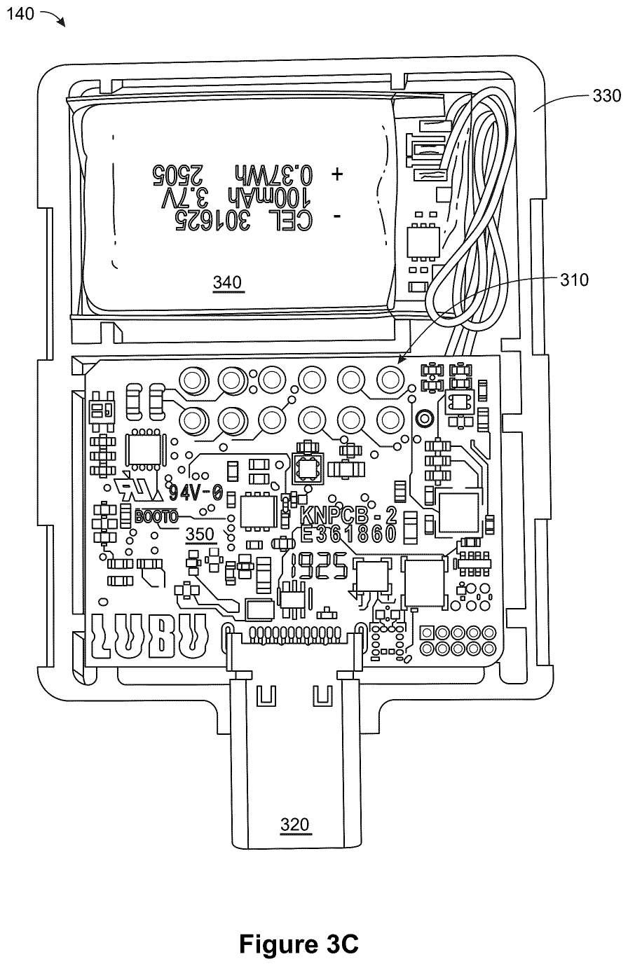

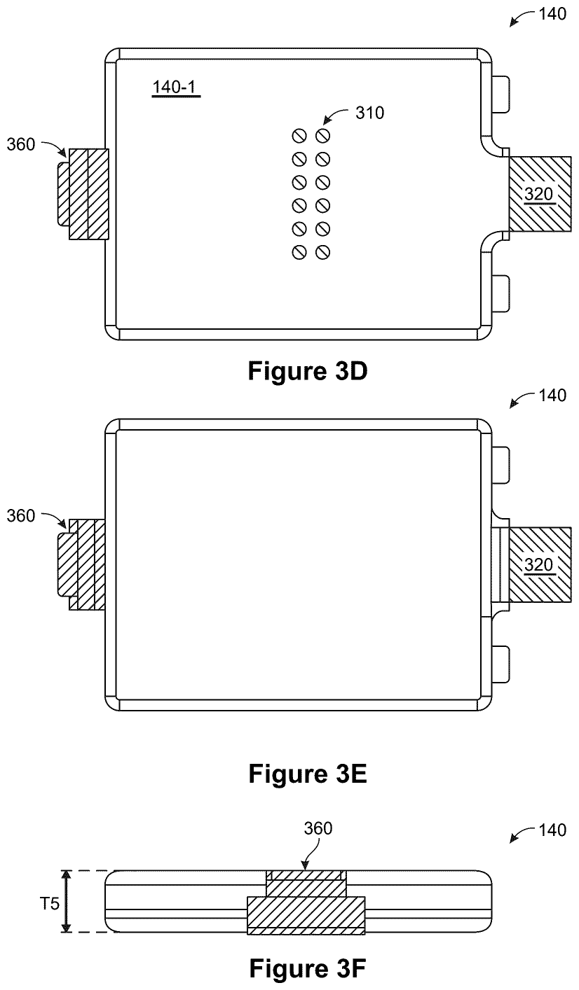

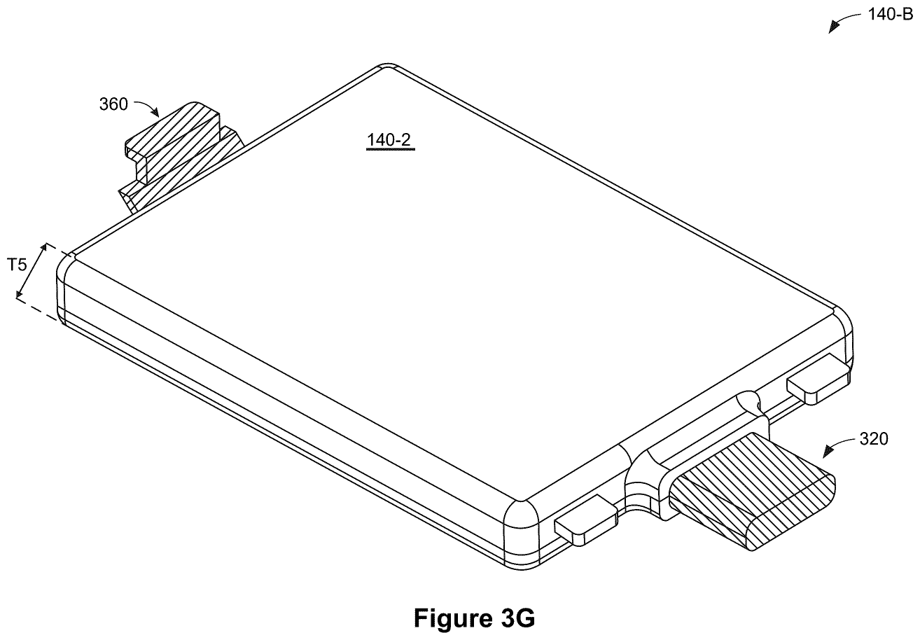

In some embodiments, the detachable electronic device 140 has a housing (e.g., the housing 330 of the detachable electronic device 140, described below with respect to FIG. 3C), which is composed of a hard material, such as a thermoplastic material, or a composite fiber material. In some embodiments, the detachable electronic device 140 is composed of a material having high tensile strength, high impact resistance, and/or high thermal resistance. In some embodiments, the housing of the detachable electronic device 140 is composed of a composite material that includes carbon fiber to reduce the weight of the detachable electronic device 140 while maintaining structural stiffness, strength, and impact resistance of the housing. In some embodiments, the housing of the detachable electronic device 140 is composed of a composite material that includes a thermoplastic (such as Nylon 12 or PA12) and carbon fibers. In some embodiments, the housing of the detachable electronic device 140 is composed of a material that includes polyoxymethylene (POM). In some embodiments, the material of the housing of the detachable electronic device 140 is chosen to provide high durability to the detachable electronic device 140 even under high mechanical stress (e.g., during athletic activity).

In some embodiments, the biomechanical assessment system 100 includes an over-mold that covers the housing 330 of the detachable electronic device 140 and is bonded in gradient to the transitional layer 120. In some embodiments, the over-mold is composed of a material that is softer (and in some embodiments, less dense) than the material of the housing 330 of the detachable electronic device 140. For example, the over-mold is composed of a material that is softer than the material of the housing 330 of the detachable electronic device 140 by at least any of 25 Shore A, 30 Shore A, 35 Shore A, and 40 Shore A.

In some embodiments, the transitional layer 120 is composed of a material that is softer and less dense compared to the material of the housing of the detachable electronic device 140. In some embodiments, the transitional layer 120 is composed of a rubber-like material, such as ethylene-vinyl acetate (EVA), which is softer and more flexible than the material of the housing of the detachable electronic device 140. In some embodiments, the transitional layer 120 is composed of a material that has a hardness anywhere between Shore A 20 and Shore 45). In some embodiments, the transitional layer 120 is composed of EVA Shore A 35 or a material having similar characteristics and/or similar density.

In some embodiments, the base layer 110 is composed of a material that has a density that is between the density of the material of the housing of the detachable electronic device 140 and the density of the material of the transitional layer 120. In some embodiments, the base layer is composed of a material that is flexible and has good resistance to impact and abrasion, such as thermoplastic polyurethane (TPU). In some embodiments, the base layer 110 is composed of TPU Shore 60 or a material having similar characteristics and/or similar density.

The material of the base layer 110 and the transitional layer 120 are designed to conceal (e.g., hide) the hardness of the housing of the detachable electronic device 140 to the underfoot of a wearer's foot. In some embodiments, the combination of the materials selected for the housing for the detachable electronic device 140, the transitional layer 120, and the base layer 110 work synergistically (e.g., collaboratively or in a complementary manner) to provide support for the wearer's foot as well as sensorially conceal the presence of the detachable electronic device 140 underfoot.

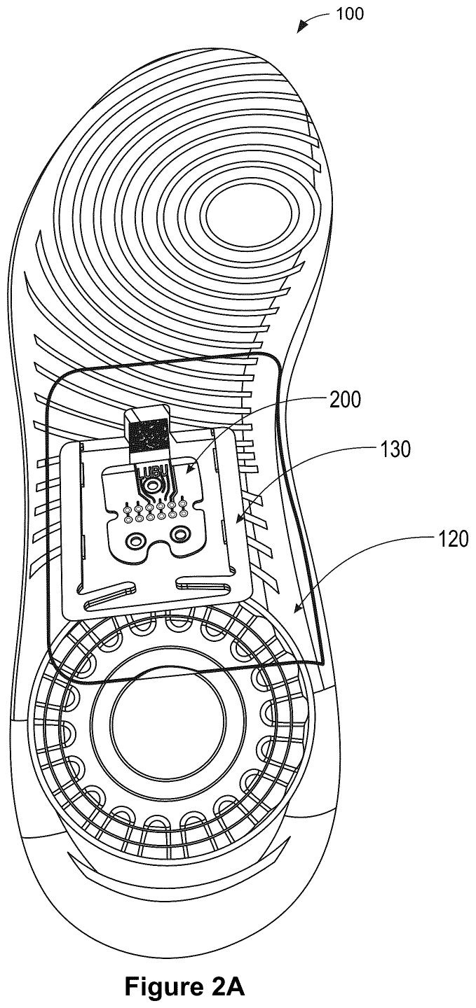

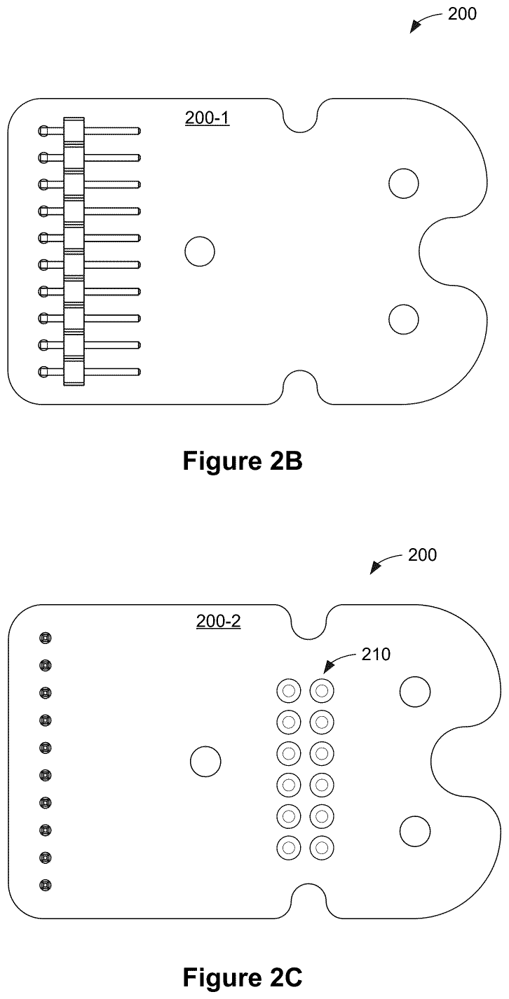

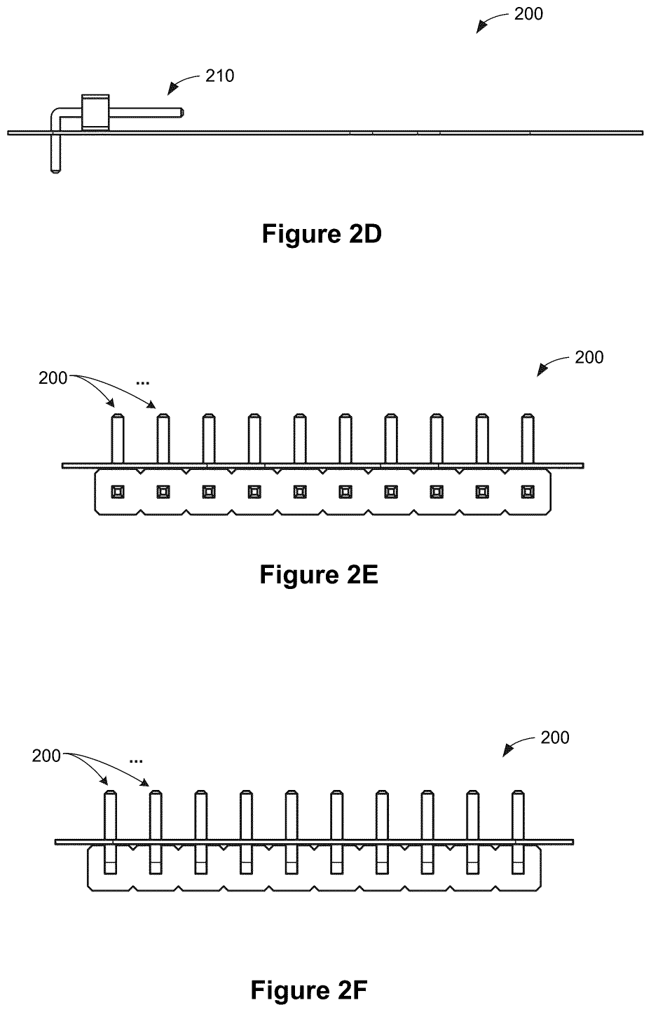

FIG. 1H also shows an electronics layer, which includes a printed circuit board 200, sensors 1100, and cables 1102. These are described below with respect to FIGS. 2A, 8A, and 11B.

In some embodiments, a cover layer 150 is located above the rest of the components, as shown in FIG. 1C.

FIG. 2A is a bottom perspective view of the portable biomechanical assessment system 100 shown in FIG. 1A, with the detachable electronic device removed, in accordance with some embodiments. As shown, the portable biomechanical assessment system 100 includes a flexible printed circuit board (PCB) 200, which is positioned inside the cut-out 120-3 of the transitional layer 120 so that the top surface 200-1 of the PCB 200 is facing a portion of the second surface 110-2 of the base layer 110 that corresponds to the cavity 110-3.

FIGS. 2B-2F are various views of the PCB 200 in accordance with some embodiments.

FIG. 2B illustrates a top view of the PCB 200 shown in FIG. 2A, showing the top surface 200-1 of the PCB 200 that is configured to be adjacent to (e.g., facing) a portion of the second surface 110-2 of the base layer 110 corresponding to the cavity 110-1.

FIG. 2C is a bottom view of the PCB 200 shown in FIG. 2A, showing a bottom surface 200-2 of the PCB 200, which is opposite from the top surface 200-1 of the PCB 200. The bottom surface 200-2 of the PCB 200 includes one or more connector pins 210, which are configured to allow the PCB 200 to electrically couple to other devices, such as the detachable electronic device 140. In some embodiments, the one or more connector pins 210 include spring-loaded pins, such as pogo pins, which are configured to maintain electrical connection (and physical contact) in high movement and high impact situations where mechanical shock and/or vibrations are expected.

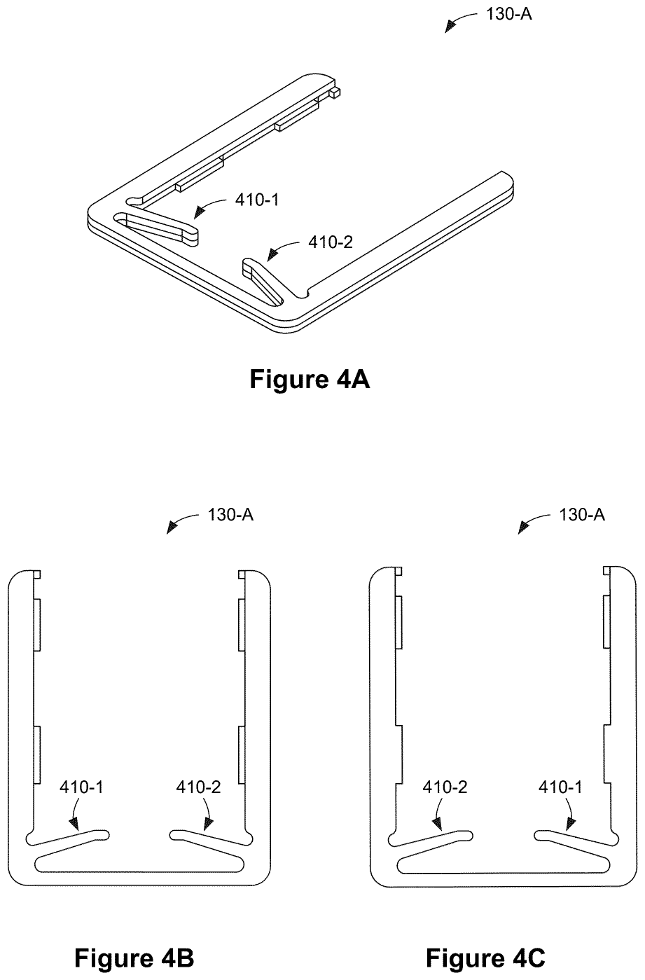

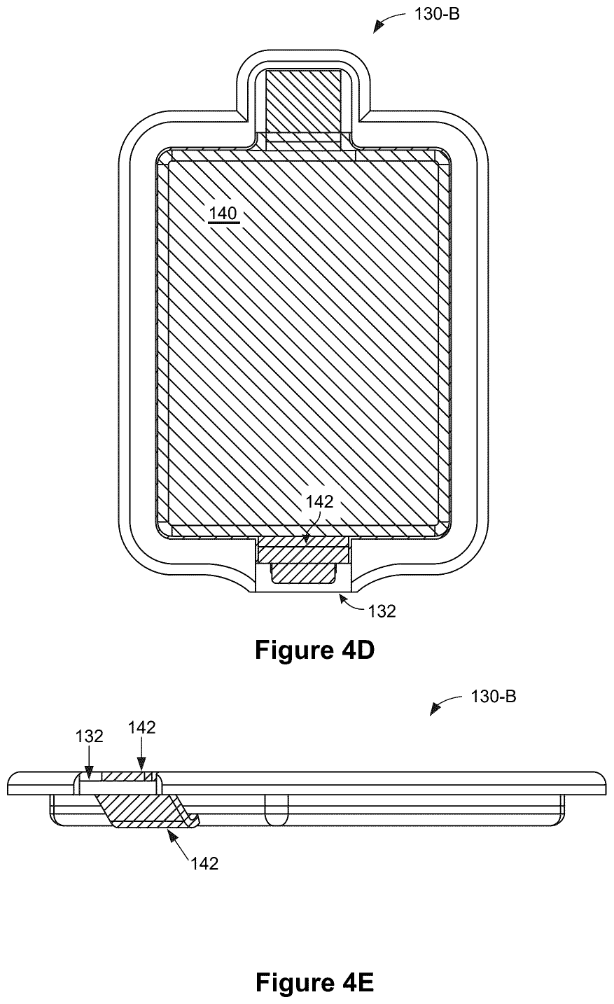

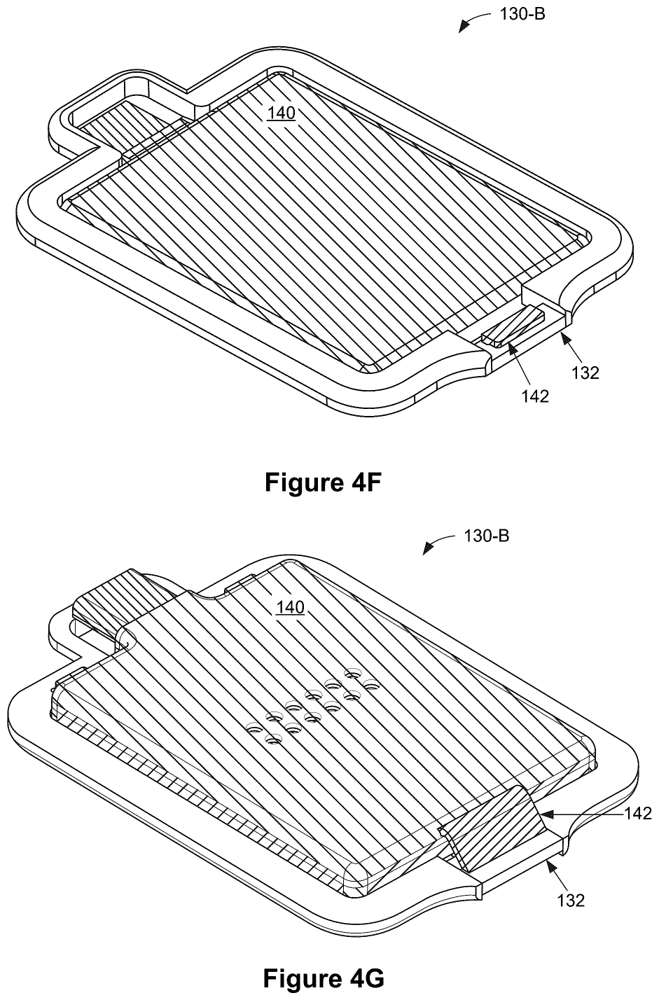

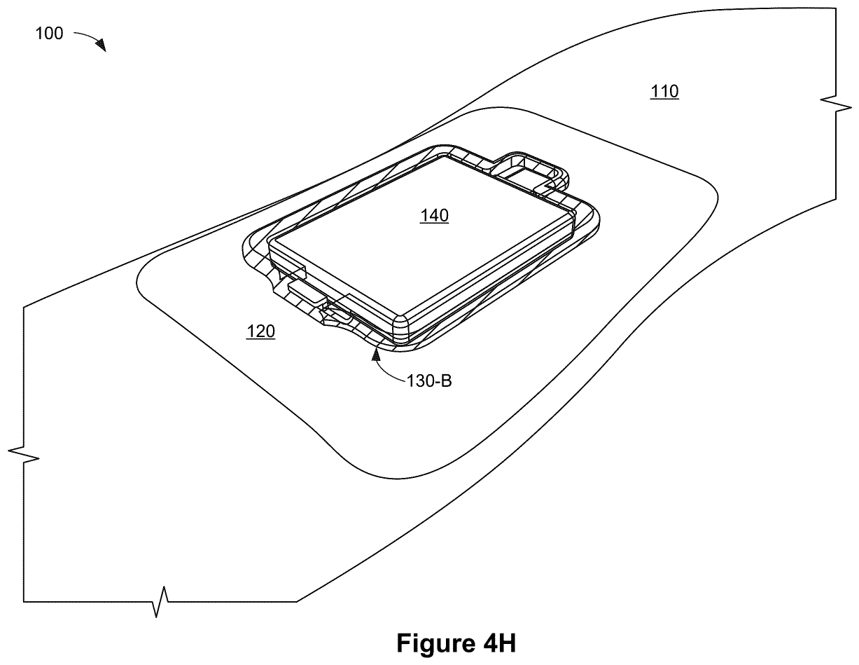

In some embodiments, the device frame 130 is configured to secure the position of an inserted detachable electronic device 140 so that one or more connectors of the detachable electronic device 140 are aligned with the one or more connectors 210 of the PCB 200. The detachable electronic device 140 is thus electrically coupled with the PCB 200. Additional details regarding the detachable electronic device 140 and the device frame 130 are provided below with respect to FIGS. 3A-3C and 4A-4H.

In some embodiments, the PCB 200 stores metadata related to the portable biomechanical assessment system 100, such as which foot a specific portable biomechanical assessment system is designed for (e.g., data corresponding to a footedness of the specific portable biomechanical assessment system). For example, a user of the portable biomechanical assessment system 100 who has two feet may require two portable biomechanical assessment systems, including one for their right foot and one for their left foot, in order to acquire complete information for movement monitoring and analysis. In this example, the PCB disposed in the portable biomechanical assessment system designed for a right foot may store information indicating that the portable biomechanical assessment system is a right-footed portable biomechanical assessment system. Similarly, the PCB disposed in the portable biomechanical assessment system designed for a left foot may store information indicating that the portable biomechanical assessment system is a left-footed portable biomechanical assessment system. By storing footedness data in the PCB 200, a detachable electronic device 140, when inserted into a device frame 130 for the portable biomechanical assessment system 100, can automatically (e.g., without manual intervention or without user configuration) determine which foot (e.g., the left foot or the right foot) the detachable electronic device 140 is currently monitoring and correlate any collected data with the correct foot.

FIGS. 2D, 2E, and 2F are a side view, a back view, and a front view of the PCB 200, respectively. The PCB 200 includes one or more connector pins 210 that are configured to form an electrical connection (and a physical connection) to connector pins of the detachable electronic device 140 (e.g., one or more device connector pins 310 of the detachable electronic device 140, described below with respect to FIG. 3A). When the detachable electronic device 140 is inserted, the information can be shared (e.g., transferred or transmitted) from the PCB 200 to the detachable electronic device 140. In some embodiments, the one or more connector pins 210 include spring-loaded electrical connectors (e.g., a plunger, barrel, or spring, also referred to as pogo pins). The spring-loaded electrical connectors are configured to establish and maintain a reliable electrical connection with the connector pins of the detachable electronic device 140 even when the portable biomechanical assessment system 100 is experiencing mechanical shock (such as during use while a wearer is engaging in sports, such as soccer).

FIGS. 3A-3C show the detachable electronic device 140 of the portable biomechanical assessment system 100 shown in FIG. 1A in accordance with some embodiments. The detachable electronic device 140 is configured to be used with device frame 130, described below with respect to FIGS. 4A-4C.

FIG. 3A is a top perspective view of the detachable electronic device 140, showing a top surface 140-1 of the detachable electronic device 140. The top surface 140-1 of the detachable electronic device 140 is configured to be adjacent to the second surface 200-2 of the PCB 200 when the detachable electronic device 140 is inserted into the device frame 130. The top surface 140-1 of the detachable electronic device 140 includes one or more device connector pins 310, which are configured to allow the detachable electronic device 140 to electrically couple to other devices, such as the PCB 200. In some embodiments, the one or more device connector pins 310 include spring-loaded pins, such as pogo pins, which are configured to maintain electrical connection (and physical contact) in high movement and high impact situations where mechanical shock and/or vibrations are expected.

The detachable electronic device 140 also includes a connector 320, which allows the detachable electronic device 140 to connect to other devices when not inserted into the device frame 130. In this example, the detachable electronic device 140 includes a universal serial bus connector (USB) type C (e.g., USB-C) connector. However, some embodiments use other connector, such as a USB-A connector or a micro-USB connector. The connector 320 is configured to allow the detachable electronic device 140 to communicatively connect to other devices and share or transmit information. For example, the detachable electronic device 140 can be connected to a computer via the connector 320 to transmit information gathered by the portable biomechanical assessment system 100 during use for analysis and storage by the computer.

FIG. 3B is a bottom view of the detachable electronic device 140, showing a bottom surface 140-2 of the detachable electronic device 140, which is opposite to the top surface 140-1 of the detachable electronic device 140. When inserted into the device frame 130, the bottom surface 140-2 of the detachable electronic device 140 forms part of the bottom surface of the foot orthotic (e.g., insole) that is the portable biomechanical assessment system 100.

FIG. 3C is a bottom view of the detachable electronic device 140 with a portion of the housing (e.g., a portion of the housing corresponding to the bottom surface 140-2) removed, showing components inside the detachable electronic device 140. The detachable electronic device 140 includes one or more connector pins 310, a connector 320, a housing 330 (e.g., shell) that houses (e.g., contains or holds) the components of the detachable electronic device 140, a battery 340, and a circuit board 350. In some embodiments, the housing 330 of the detachable electronic device 140 is configured to encase and protect components of the detachable electronic device 140 (e.g., the circuit board 350 and the battery 340).

The circuit board 350 includes (e.g., has electrical connections to) the one or more connector pins 310 and the connector 320. The battery 340 is configured to provide power to the circuit board 350. In some embodiments, the detachable electronic device 140 also includes one or more inertial measurement units (such as an accelerometer and/or a gyroscope). In some embodiments, the one or more inertial measurement units are included as part of the circuit board 350.

FIGS. 3D, 3E, 3F, and 3G are a bottom view, a top view, a side view, and a perspective top view of a detachable electronic device 140 of the portable biomechanical assessment system 100 shown in FIG. 1A in accordance with some embodiments. The detachable electronic device 140 is configured to be used with device frame 130, described below with respect to FIGS. 4D-4H.

The detachable electronic device 140 is similar to the detachable electronic device 140 described above with respect to FIGS. 3A-3C, with the addition of a protruding member 360, which allows the detachable electronic device 140 to be secured by the device frame 130 via a snap-fit mechanism. As shown, the detachable electronic device 140 includes a protruding member 360, which is configured to mate with (e.g., snap into) a snap-in region of device frame 130 so that the device frame 130 can secure (e.g., hold or lock) the detachable electronic device 140 in place.

As shown in FIGS. 3F and 3G, the detachable electronic device 140 has a thickness T5. In some embodiments, the thickness T5 that is 5.0 mm, 5.5 mm, 5.8 mm, or 6.0 mm.

FIGS. 4A-4C illustrate a first embodiment 130-A of the device frame 130 of the portable biomechanical assessment system 100 shown in FIG. 1A, in accordance with some embodiments. FIGS. 4A, 4B, and 4C provide a perspective view, a bottom view, and a top view of the device frame 130-A, respectively. The device frame 130-A utilizes a flex-fit mechanism (also referred to as a preload flex fit) to secure the detachable electronic device 140 in position. The device frame 130-A includes flexible members 410-1 and 410-2, which are configured to hold the detachable electronic device 140 securely in position when the detachable electronic device 140 is inserted into the device frame 130-A (e.g., so that the device connector pins 310 of the detachable electronic device 140 are in alignment with and in contact with the connector pins of the PCB 200). The flexible members 410-1 and 410-2 are configured to apply force against a portion of the detachable electronic device 140 so that the device frame 130-A surrounds at least a portion of the detachable electronic device 140 when the detachable electronic device 140 is inserted.

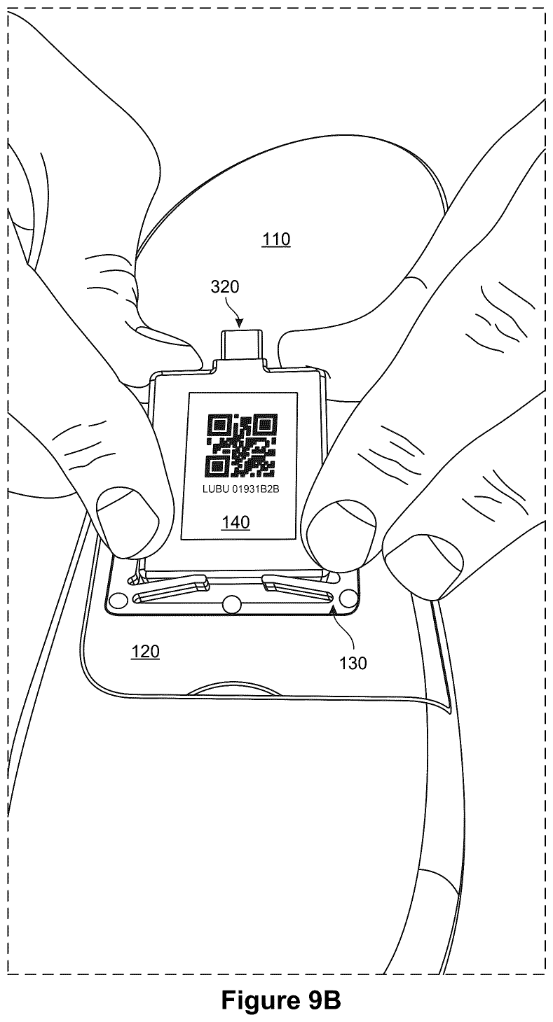

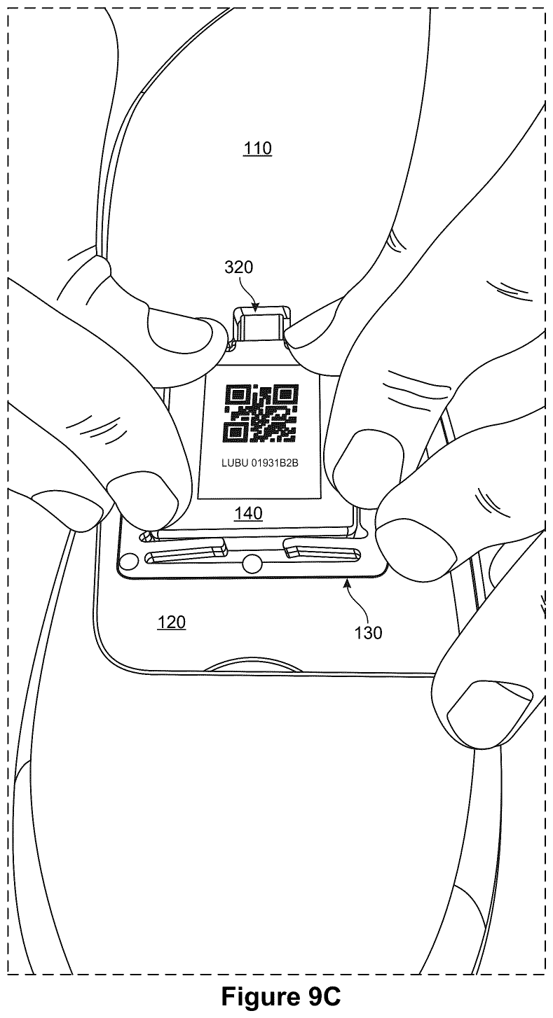

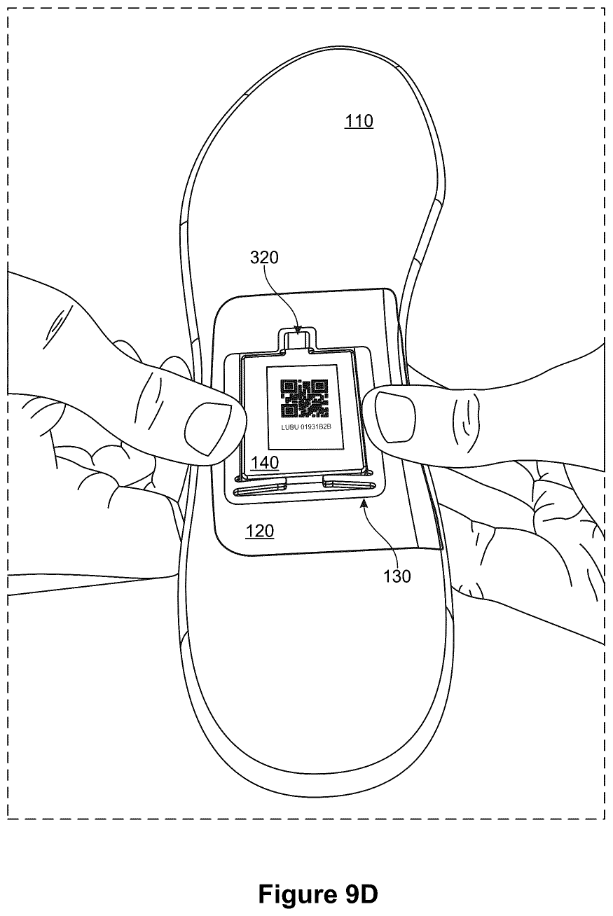

An example of how to insert and remove the detachable electronic device 140 from the device frame 130-A is provided below with respect to FIG. 9A-9D.

FIGS. 4D-4H illustrate a second embodiment 130-B of the device frame 130 of the portable biomechanical assessment system 100 shown in FIG. 1A, in accordance with some embodiments. FIGS. 4D-4H illustrate a bottom view, a perspective side view, a bottom perspective view, a top perspective view, and a bottom context view of the device frame 130-B with detachable electronic device 140-B inserted. The device frame 130-B utilizes a snap-fit mechanism to secure the detachable electronic device 140-B in position. The device frame 130-B has a snap-in region 132 that is configured to fasten the detachable electronic device 140 securely in position when the detachable electronic device 140 is inserted into the device frame 130-B (e.g., so that the device connector pins 310 of the detachable electronic device 140 are in alignment with and in contact with the connector pins of the PCB 200). The snap-in region 132 is configured to allow a protruding member 360 of the detachable electronic device 140 to be fastened at the snap-in region 132 so that the device frame 130-B surrounds at least a portion of the detachable electronic device 140 when the detachable electronic device 140 is inserted.

The detachable electronic device 140 can be inserted into the device frame 130-B by gently pressing the detachable electronic device 140 downwards into the cutout formed by the device frame 130-B and the transitional layer 120. The protruding member 360 of the detachable electronic device 140 will snap onto (e.g., latch onto) a snap-in region 132 of the device frame 130-B, thereby securing the detachable electronic device 140 in place. To remove the detachable electronic device 140, a user gently presses on a portion of the detachable electronic device 140 (e.g., a lever 142) that is connected to the protruding member 360 so that the protruding member 360 retracts away from the snap-in region 132 of the device frame 130-B, thereby releasing the snap-fit connection (e.g., the latch) between the protruding member 360 of the detachable electronic device 140 and the snap-in region 132 of the device frame 130-B.

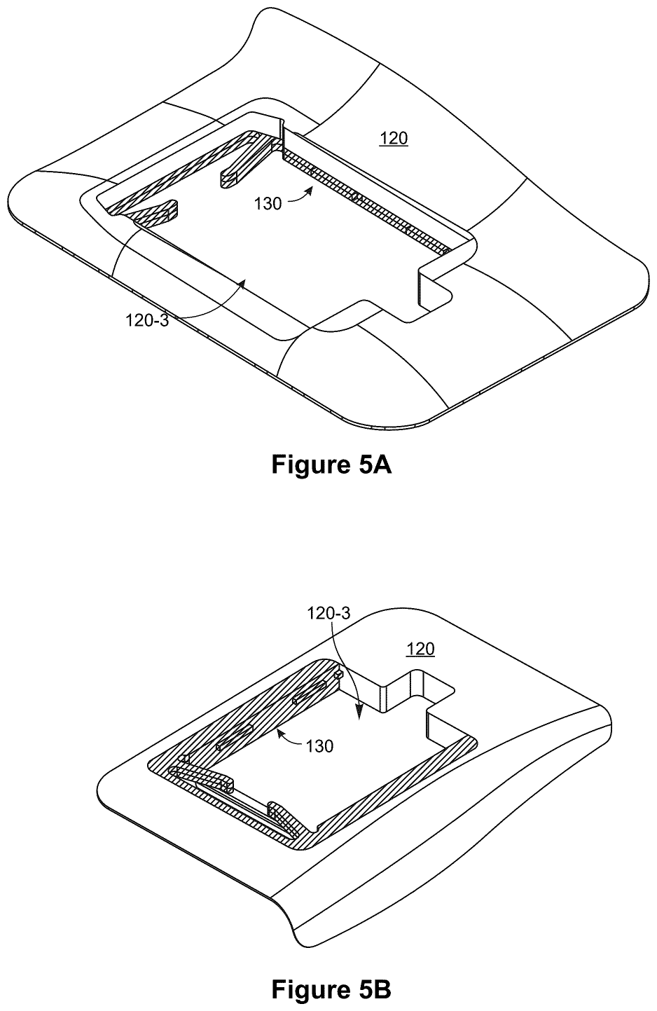

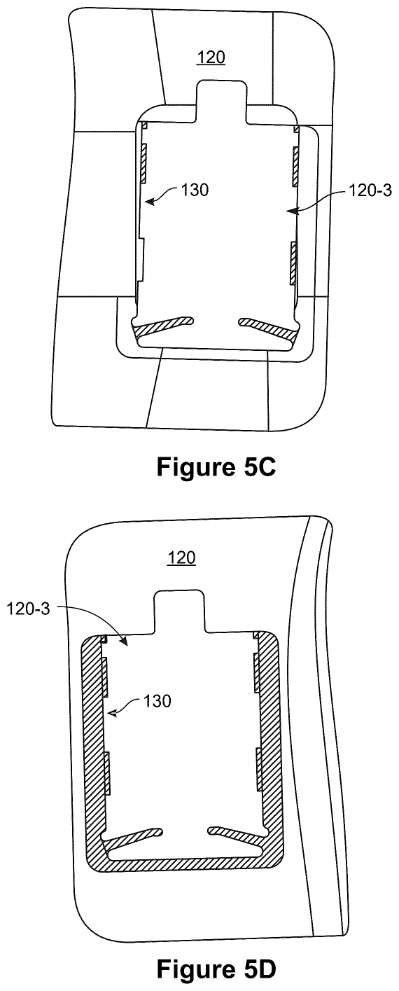

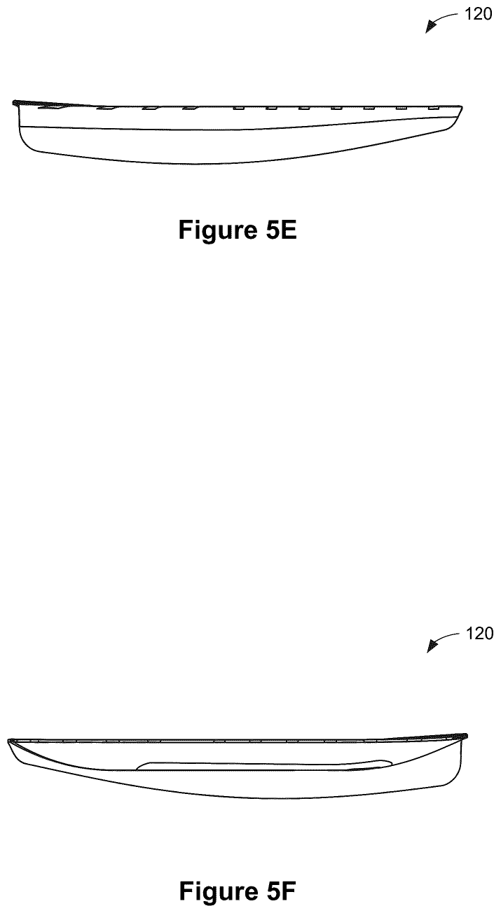

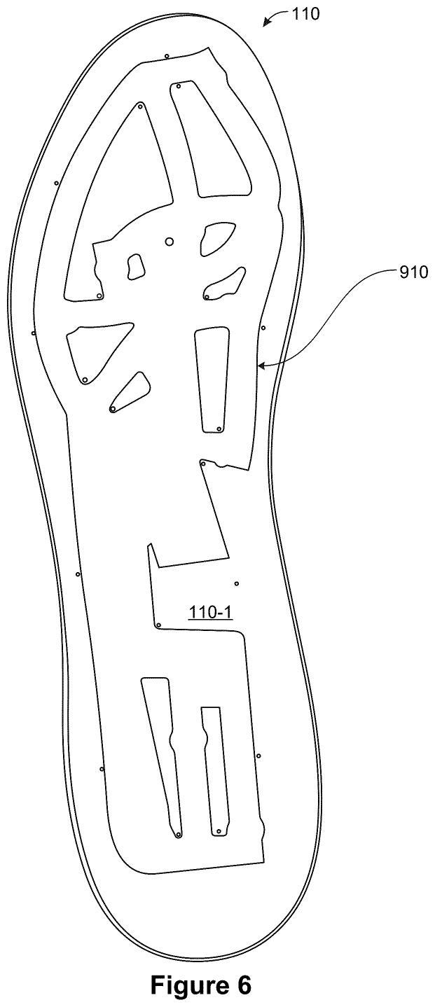

FIGS. 5A-5F show a top perspective view, a bottom perspective view, a top view, a bottom view, a medial side view, and a lateral side view of the device frame 130 (shown filled in with a diagonal pattern) and the transitional layer 120 of the portable biomechanical assessment system 100 shown in FIG. 1A, in accordance with some embodiments.

The transitional layer 120 includes a cut-out 120-3, which is configured to accommodate the device frame 130 (including either the first device frame embodiment 130-A or the second device frame embodiment 130-B). The transitional layer 120 is composed of a material that is softer and more flexible than the material of the device frame 130. The material of the transitional layer 130 is chosen to mitigate the sensation of the device frame 130 and the detachable electronic device 140. Thus, the transitional layer 130 is configured to provide support to the midfoot portion of the wearer's foot (including the wearer's arch) as well as improve the underfoot sensation for the wearer by masking the hardness and the presence of the device frame 130 and the detachable electronic device 140.

FIG. 6 illustrates the base layer 110 of the portable biomechanical assessment system 100 shown in FIG. 1A, in accordance with some embodiments. The base layer 110 includes a first surface 110-1 (visible in FIG. 6) and a second surface 110-2 (on the opposite, and thus not visible in FIG. 6). The second surface 110-2 includes a cavity 110-3 that is configured to accommodate the transitional layer 120, the device frame 130, and the detachable electronic device 140 (when inserted) such that the base layer 110 at least partially surrounds the transitional layer 120, the device frame 130, and the detachable electronic device 140.

In some embodiments, the first surface 110-1 of the base layer 110 includes a plurality of grooves 910, which are configured to accommodate one or more sensors and any cables required to connect (e.g., couple, electrically couple, and/or communicatively couple) the one or more sensors to the PCB 200. Each of the sensors and each of the cables is disposed in the grooves 910 so that the sensors and cables are embedded in the base layer 110. In some embodiments, the grooves 910 have a depth that is at least equal to a thickness of the sensors and the cables so that a wearer of the portable biomechanical assessment system 100 cannot feel the sensors or cables protruding out of the portable biomechanical assessment system 100. In some embodiments, the grooves 910 have a depth that is the same as or greater than the thickness of the plurality of sensors by no more than a predetermined amount, so that the plurality of sensors can sense pressure changes from the wearer's foot. In some embodiments, the predetermined amount is 0.01 mm, 0.04 mm, 0.07 mm, 0.1 mm, or 0.2 mm.

Additional details regarding the sensors and cables are provided below with respect to FIGS. 8A-8B.

In some embodiments, the base layer 110 is configured to work synergistically (e.g., in combination with) the transitional layer 120 to mitigate (e.g., mask) the hardness of the device frame 130 and the detachable electronic device 140 for the sole of the wearer's foot. In some embodiments, the base layer 110 is composed of a material that has a hardness that is greater than the hardness of the material of the transitional layer 120. In some embodiments, the base layer 110 is composed of a material that is more dense than the density of the material of the transitional layer 120. In some embodiments, the base layer 110 is composed of a material that has a hardness that is less than the hardness of the material of the device frame 130 and the hardness of the detachable electronic device 140. In some embodiments, the base layer 110 is composed of a material that has a density that is less than the density of the material of the device frame 130 and the density of the detachable electronic device 140.

In some embodiments, the base layer 110 is configured to provide structure and support to the foot orthotic (the portable biomechanical assessment system 100) while masking the presence of the device frame 130 and the detachable electronic device 140 for the underfoot sensations of a wearer of the portable biomechanical assessment system 100.

In some embodiments, the base layer 110 includes one or more contours that are configured to reduce the bulk and profile of the foot orthotic so that the portable biomechanical assessment system 100 can be used with a shoe that requires a low-profile insole, such as an athletic cleat (e.g., a soccer shoe).

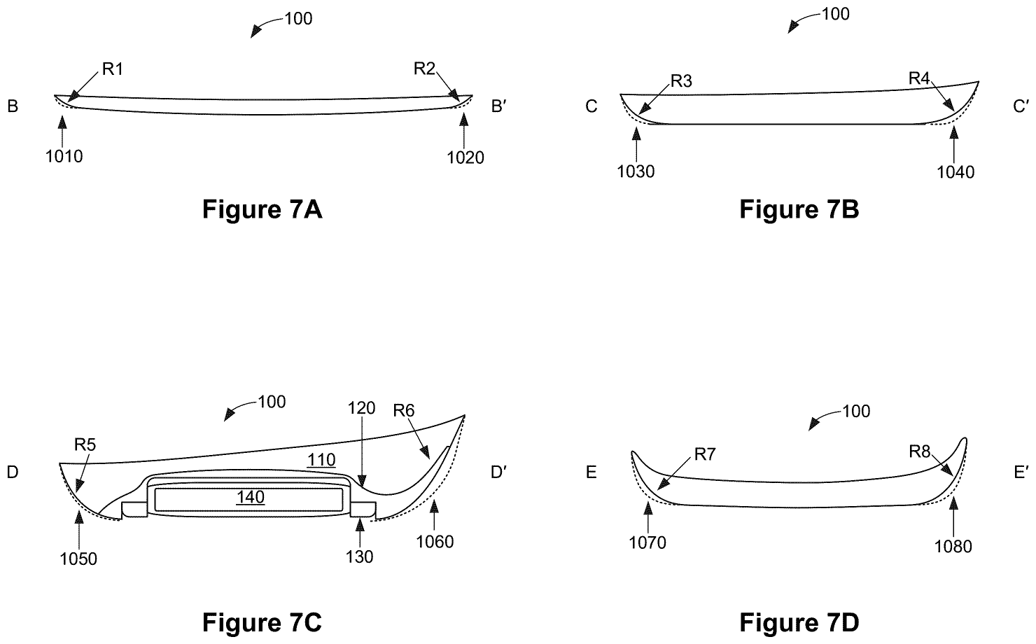

FIGS. 7A to 7D are cross-sectional views of the portable biomechanical assessment system 100, showing the shapes and contours of the foot orthotic in accordance with some embodiments.

FIG. 7A is a cross-sectional view of the portable biomechanical assessment system 100 across BB′, shown in FIG. 1F. The outside edge (e.g., perimeter) of the portable biomechanical assessment system 100 is defined by the shape of the base layer 110 along the forefoot and heel portions and is defined by the shape of the base layer 110 and the transitional layer 120 along the midfoot portion. The outside edge of the portable biomechanical assessment system 100 includes a first contour 1010 along a portion of a lateral edge corresponding to forefoot portion of the portable biomechanical assessment system 100 and a second contour 1020 along a portion of a medial edge corresponding to forefoot portion of the portable biomechanical assessment system 100. The first contour 1010 has a first radius of curvature R1 and the second contour 1020 has a second radius of curvature R2. The first radius of curvature R1 and the second radius of curvature R2 are configured to reduce the profile and bulk of the portable biomechanical assessment system 100 compared to traditional insoles. An example profile of a traditional insole having a contour with a radius of curvature of ˜3 mm is shown in FIG. 7A as a dotted line for comparison. In some embodiments, the first contour 1010 has a first radius of curvature R1 that is 3.5 mm, 5 mm, 7 mm, or 8 mm. In some embodiments, the second contour 1020 has a second radius of curvature R2 that is 3 mm, 4 mm, 5 mm, 7 mm, and 8 mm.

FIG. 7B is a cross-sectional view of the portable biomechanical assessment system 100 across CC′, shown in FIG. 1F. The outside edge of the portable biomechanical assessment system 100 includes a third contour 1030 along a portion of a lateral edge corresponding to portion of the portable biomechanical assessment system 100 where the forefoot and midfoot connect (e.g., near the tarsometatarsal joints (TMTJs)) and a fourth contour 1040 along a portion of a medial edge corresponding to portion of the portable biomechanical assessment system 100 where the forefoot and midfoot connect (e.g., near the TMTJs). The third contour 1030 has a third radius of curvature R3 and the fourth contour 1040 has a fourth radius of curvature R4. The third radius of curvature R3 and the fourth radius of curvature R4 are configured to reduce the profile and bulk of the portable biomechanical assessment system 100 compared to traditional insoles. An example profile of a traditional insole having a contour with a radius of curvature of ˜5 mm is shown in FIG. 7B as a dotted line for comparison. In some embodiments, the third contour 1030 has a third radius of curvature R3 that is larger than any of: 6 mm, 7 mm, 8 mm, and 9 mm. In some embodiments, the fourth contour 1040 has a fourth radius of curvature R4 that is 5 mm, 7 mm, 9 mm, 11 mm, or 12 mm.

FIG. 7C is a cross-sectional view of the portable biomechanical assessment system 100 across DD′, shown in FIG. 1F. The outside edge of the portable biomechanical assessment system 100 includes a fifth contour 1050 along a portion of a lateral edge corresponding to portion of the portable biomechanical assessment system 100 where the midfoot and hindfoot connect (e.g., near the Chopart joint) and a sixth contour 1060 along a portion of a medial edge corresponding to portion of the portable biomechanical assessment system 100 where the midfoot and the hindfoot connect (e.g., near the Chopart joint). The fifth contour 1050 has a fifth radius of curvature R5 and the sixth contour 1060 has a sixth radius of curvature R6. The fifth radius of curvature R5 and the sixth radius of curvature R6 are configured to reduce the profile and bulk of the portable biomechanical assessment system 100 compared to traditional insoles. An example profile of a traditional insole having a lateral contour with a radius of curvature of ˜9 mm and a medial contour with a radius of curvature of ˜14 mm is shown in FIG. 7C as dotted lines for comparison. In some embodiments, the fifth contour 1050 has a fifth radius of curvature R5 that is 9.0 mm, 9.2 mm, 9.5 mm, 9.7 mm, or 10.0 mm. In some embodiments, the sixth contour 1040 has a sixth radius of curvature R6 that is 12 mm, 12.5 mm, 13 mm, 13.5 mm, or 14 mm.

The medial edge of the portable biomechanical assessment system 100 shown in FIG. 7C has a larger profile (e.g., is taller, is thicker, and/or has a raised edge that comes up higher) than the lateral edge of the portable biomechanical assessment system 100 shown in FIG. 7C.

FIG. 7D is a cross-sectional view of the portable biomechanical assessment system 100 across EE′, shown in FIG. 1F. The outside edge of the portable biomechanical assessment system 100 includes a seventh contour 1070 along a portion of a lateral edge corresponding to a hindfoot (e.g., heel) portion of the portable biomechanical assessment system 100 and an eighth contour 1080 along a portion of a medial edge corresponding to hindfoot (e.g., heel) portion of the portable biomechanical assessment system 100. The seventh contour 1070 has a seventh radius of curvature R7 and the eighth contour 1080 has an eighth radius of curvature R8. The seventh radius of curvature R7 and the eighth radius of curvature R8 are configured to reduce the profile and bulk of the portable biomechanical assessment system 100 compared to traditional insoles. An example profile of a traditional insole having a lateral contour and a medial contour corresponding to a hindfoot portion of the insole with a radius of curvature of ˜7 mm is shown in FIG. 7D as dotted lines for comparison.

In some embodiments, the seventh contour 1050 has a seventh radius of curvature R7 that is 7.0 mm, 8.0 mm, 9.0 mm, 10 mm, 11 mm, or 12.0 mm. In some embodiments, the eighth contour 1080 has an eighth radius of curvature R8 that is 7.0 mm, 8.0 mm, 9.0 mm, 10 mm, 11 mm, or 12.0 mm.

FIGS. 8A and 8B show sensors 1100 and cables 1102 of the portable biomechanical assessment system 100 shown in FIG. 1A, in accordance with some embodiments.

In some embodiments, the portable biomechanical assessment system 100 includes one or more sensors 1100 and one or more cables 1102 that are configured to connect (e.g., couple, electrically couple, and/or communicatively couple) the one or more sensors 1100 to the PCB 200. In some embodiments, the one or more sensors 1100 include at least one pressure sensor that is configured to output an electrical signal that is proportional to the force (pressure) exerted (e.g., by the wearer's foot) on the pressure sensor. In some embodiments, the one or more sensors 1100 include at least one temperature sensor configured to output an electrical signal that is proportional to the change in temperature at the temperature sensor.

The electrical signal output from the one or more sensors 1100 is transmitted, via the cables 1102 to the PCB 200. When the PCB 200 is connected (e.g., electrically connected or physically connected) to the detachable electronic device 140 (e.g., the circuit board 350 of the PCB 200, via the connector pins 210 and 310), the electrical signal output from the one or more sensors 1100 are transmitted to the detachable electronic device 140 and stored at the detachable electronic device 140 as sensor data until the detachable electronic device 140 is removed and connected to (e.g., plugged into or via the connector 320) another device that can store and analyze the sensor data.

In some embodiments, each of the sensors has a thickness that is no greater than 0.3 mm, 0.33 mm, 0.36 mm, 0.4 mm, 0.45 mm, or 0.5 mm.

In some embodiments, the biomechanical assessment system 100 is configured to support additional sensors (e.g., sensing modules), such as, but not limited to: hydration sensor(s), heart rate sensor(s), electromyography (EMG) sensor(s), location sensor(s) (such as a global positioning system (GPS) device), and position sensor(s) (such as accelerometers and gyroscopes).

FIGS. 9A-9C show how to insert the detachable electronic device 140 into the device frame 130 in accordance with some embodiments.

FIG. 9D shows how to remove the detachable electronic device 140 from the device frame 130 in accordance with some embodiments. Users apply a gentle force downwards using their thumbs and a gentle upward force using their fingers to slightly bend the foot orthotic in the side-to-side (e.g., left-right) direction, causing the detachable electronic device 140 to pop out of the device frame 130. The detachable electronic device 140 can now be connected to a computing device (e.g., a smart phone, a tablet, or a computer) to transfer data (e.g., IMU data and/or sensor data) stored in the detachable electronic device 140 to the computing device and perform analysis on the data acquired from the detachable electronic device 140.

The detachable electronic device 140 is configured to be usable with any composite core 101. For example, at a first time, a detachable electronic device 140 may be inserted into a first insole (e.g., into the composite core 101) that is designed to be used (e.g., worn) in the left foot of a soccer cleat. The first insole and the detachable electronic device 140 are in communication with one another and can collect, store, and transmit data collected while a user is wearing the soccer cleat (with the first insole and the detachable electronic device 140 inserted). Following this example, at a second time that is different from the first time, the user removes the detachable electronic device 140 from the first insole and inserts the same detachable electronic device 140 into a second insole that is different from the first insole. For example, the second insole (e.g., another composite core 101) may be an insole that is designed to be used (e.g., worn) in the right foot of a work boot. The second insole and the detachable electronic device 140 are in communication with one another and can collect, store, and transmit data collected while a user is wearing the work boot (with the second insole and the detachable electronic device 140 inserted).

In another example, as the support layers of the composite core 101 degrade in performance over time, a user may purchase a replacement composite core 101 that is compatible with the existing detachable electronic device 140 without needing to purchase a new detachable electronic device 140.

Thus, the detachable electronic device 140 is modular and can be quickly swapped between different insoles (e.g., composite cores 101). The detachable electronic device 140 can also seamlessly connect to and integrate with any insole (e.g., composite core 101) that is designed for use with the detachable electronic device 140.

Turning now to some example embodiments.

In one aspect, some embodiments include a foot orthotic 100, which includes a device frame 130 configured to secure a detachable electronic device 140, and a composite core 101. The composite core includes a transitional layer 120 and a base layer 110. The transitional layer includes a cut-out 120-3, which is configured to accommodate the device frame and the detachable electronic device. The base layer at least partially surrounds the transitional layer and includes a cavity 110-3 configured to accommodate the transitional layer.

(A2) In some embodiments, the foot orthotic of A1 further comprises a printed circuit board 200, which is disposed inside the cut-out of the transitional layer. The printed circuit board includes one or more first connector pins 210 that are configured to provide electrical contact with one or more second connector pins 310 of the detachable electronic device while the detachable electronic device is attached in the device frame.

(A3) In some embodiments of the foot orthotic of A2, the printed circuit board stores metadata that specifies whether the orthotic is constructed for a left shoe or a right shoe so that a detachable electronic device, while attached in the device frame, can automatically detect (e.g., without additional user configuration or input) whether the detachable electronic device has been inserted into a left-sided orthotic or a right-sided orthotic.

(A4) In some embodiments, the foot orthotic of A3 further comprises a detachable electronic device having the one or more second connector pins. The cut-out of the transitional layer is configured to accommodate the detachable electronic device. The detachable electronic device is configured to be positioned inside the cut-out by means of the device frame so that the one or more second connector pins of the detachable electronic device maintain contact with the one or more first connector pins of the printed circuit board. The detachable electronic device is configured to, upon establishing an electrical connection to the printed circuit board: read data stored in the printed circuit board and automatically (e.g., without additional user configuration or input) determine whether the detachable electronic device has been inserted into a left-sided orthotic or a right-sided orthotic based on data stored by the printed circuit board.

(A5) In some embodiments of the foot orthotic of any of A1-A4, the base layer is comprised of a material having a first density that is configured to sensorially conceal the device frame and the detachable electronic device from the sole of the user's foot while using the foot orthotic. The first density is between the density of the transitional layer and the density of the detachable electronic device. The first density is different from each of the density of the transitional layer, the density of the device frame, and the density of the detachable electronic device.

(A6) In some embodiments of the foot orthotic of A4 or A5, the detachable electronic device includes one or more inertial measurement units.

(A7) In some embodiments of the foot orthotic of any of A4-A6, the detachable electronic device is configured to wirelessly transmit a digital signal to a user device that is communicatively paired with the detachable electronic device.

(A8) In some embodiments, the foot orthotic of any of A2-A7 further comprises a plurality of sensors 1100 in the foot orthotic. The plurality of sensors is electronically coupled to the printed circuit board.

(A9) In some embodiments of the foot orthotic of A8, the plurality of sensors includes one or more pressure sensors and each of the one or more pressure sensors is configured to output a respective electronic signal in response to applied force on the respective pressure sensor.

(A10) In some embodiments of the foot orthotic of A8 or A9, the plurality of sensors includes one or more temperature sensors and each of the one or more temperature sensors is configured to output a respective electronic signal in response to changes in temperature at the respective temperature sensor.

(A11) In some embodiments of the foot orthotic of any of A8-A10, the detachable electronic device is configured to receive electronic signals output by the plurality of sensors and generate a signal based on the electronic signals while the detachable electronic device is attached in the device frame.

(A12) In some embodiments, the foot orthotic of any of A8-A11 further comprises a plurality of cables 1102 configured to electronically couple the plurality of sensors to the printed circuit board so that electrical signals output from the plurality of sensors can be electronically transmitted to the detachable electronic device via the printed circuit board when the detachable electronic device is attached in the device frame.

(A13) In some embodiments of the foot orthotic of any of A9-A12, the base layer includes a first surface 110-1 having plurality of cavities and a plurality of grooves 910. Each sensor of the plurality of sensors is positioned in a respective cavity of the plurality of cavities so that the sensors are embedded in the base layer, and each cable of a plurality of cables is positioned in a respective groove of the plurality of grooves so that the cables are embedded in the base layer.

(A14) In some embodiments of the foot orthotic of A13, the plurality of sensors has a maximum sensor thickness and the plurality of cavities has a second thickness that is greater than the maximum sensor thickness within a predetermined range so that the sole of the user's foot is in contact with the plurality of sensors while using the foot orthotic.

(A15) In some embodiments, the foot orthotic of any of A8-A14 further comprises a cover layer 150 that is disposed adjacent to the first surface 110-1 of the base layer so that at least a portion of the cover layer is in physical contact with the plurality of sensors. The cover layer is configured to be adjacent to the sole of the user's foot while using the foot orthotic.

(A16) In some embodiments of the foot orthotic of any of A1-A15, the device frame 130 includes one or more flexible members 410-1 and 410-2, which are configured to hold the detachable electronic device 140 in position. The one or more flexible members are configured to apply force against a portion of the detachable electronic device so that the device frame surrounds at least a portion of the detachable electronic device when attached.

(A17) In some embodiments of the foot orthotic of any of A1-A16, the device frame 130 includes a snap-in region 132, which is configured to fasten the detachable electronic device 140 in position when the detachable electronic device is inserted. The snap-in region is configured to allow a protruding member 360 of the detachable electronic device to be fastened at the snap-in region so that the device frame surrounds at least a portion of the detachable electronic device when the detachable electronic device is inserted.

(A18) In some embodiments of the foot orthotic of any of A1-A17, the composite core includes a first surface that is configured to be adjacent to the sole of the user while in use, a second surface that is opposite the first surface, and a raised perimeter so that the first surface includes concave contours and the second surface includes convex contours.

(A19) In some embodiments of the foot orthotic of A18, the convex contours of the second surface of the composite core have predefined radii of curvature so that the foot orthotic has a small volume along the perimeter of the composite core.

(B1) In another aspect, some embodiments include a portable biomechanical assessment system 100, which includes: a detachable electronic device 140 having one or more first connector pins 310, a device frame 130, such as the first and second embodiments 130-A and 130-B, which is configured to secure the detachable electronic device, a transitional layer 120, which includes a cut-out 120-3 configured to accommodate the device frame and the detachable electronic device, a base layer 110, which at least partially surrounds the transitional layer, and a printed circuit board 200, which is disposed inside the cut-out of the transitional layer. The base layer includes a cavity 110-3, which is configured to accommodate the transitional layer. The printed circuit board includes one or more second connector pins 210. The device frame is configured to secure the detachable electronic device in position so that electrical contact is maintained between the one or more first connector pins of the detachable electronic device and the one or more second connector pins of the printed circuit board while the detachable electronic device is attached in the device frame.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A foot orthotic, comprising:
a device frame configured to secure a detachable electronic device; and
a composite core that includes:
a transitional layer that includes a cut-out configured to accommodate the device frame and the detachable electronic device; and
a base layer that at least partially surrounds the transitional layer and includes a cavity that is configured to accommodate the transitional layer.

2. The foot orthotic of claim 1, further comprising:
a printed circuit board disposed inside the cut-out of the transitional layer, wherein the printed circuit board includes one or more first connector pins that are configured to provide electrical contact with one or more second connector pins of the detachable electronic device while the detachable electronic device is attached in the device frame.

3. The foot orthotic of claim 2, wherein:
the printed circuit board stores metadata that specifies whether the orthotic is constructed for a left shoe or a right shoe so that a detachable electronic device, while attached in the device frame, can automatically detect whether the detachable electronic device has been inserted into a left-sided orthotic or a right-sided orthotic.

4. The foot orthotic of claim 3, further comprising:
a detachable electronic device having the one or more second connector pins, wherein:
the cut-out of the transitional layer is configured to accommodate the detachable electronic device;
the detachable electronic device is configured to be positioned inside the cut-out by means of the device frame so that the one or more second connector pins of the detachable electronic device maintain contact with the one or more first connector pins of the printed circuit board; and
the detachable electronic device is configured to, upon establishing an electrical connection to the printed circuit board:
read data stored in the printed circuit board; and
automatically determine whether the detachable electronic device has been inserted into a left-sided orthotic or a right-sided orthotic based on data stored by the printed circuit board.

5. The foot orthotic of claim 4, wherein:
the base layer is comprised of a material having a first density that is configured to sensorially conceal the device frame and detachable electronic device from a sole of a user's foot while using the foot orthotic;
the first density is between a density of the transitional layer and a density of the detachable electronic device; and
the first density is different from each of the density of the transitional layer, a density of the device frame, and the density of the detachable electronic device.

6. The foot orthotic of claim 4, wherein the detachable electronic device includes one or more inertial measurement units.

7. The foot orthotic of claim 4, wherein the detachable electronic device is configured to wirelessly transmit a digital signal to a user device that is communicatively paired with the detachable electronic device.

8. The foot orthotic of claim 2, further comprising:
a plurality of sensors, in the foot orthotic, that are electronically coupled to the printed circuit board.

9. The foot orthotic of claim 8, wherein:
the plurality of sensors includes one or more pressure sensors; and
each of the one or more pressure sensors is configured to output a respective electronic signal in response to applied force on the respective pressure sensor.

10. The foot orthotic of claim 8, wherein:
the plurality of sensors includes one or more temperature sensors; and
each of the one or more temperature sensors is configured to output a respective electronic signal in response to changes in temperature at the respective temperature sensor.

11. The foot orthotic of claim 8, wherein the detachable electronic device is configured to receive electronic signals output by the plurality of sensors and generate a digital signal based on the electronic signals while the detachable electronic device is attached in the device frame.

12. The foot orthotic of claim 8, further comprising:
a plurality of cables configured to electronically couple the plurality of sensors to the printed circuit board so that electrical signals output from the plurality of sensors can be electronically transmitted to the detachable electronic device via the printed circuit board when the detachable electronic device is attached in the device frame.

13. The foot orthotic of claim 9, wherein:
the base layer includes a first surface having plurality of cavities and a plurality of grooves;
each sensor of the plurality of sensors is positioned in a respective cavity of the plurality of cavities so that the sensors are embedded in the base layer; and
each cable of a plurality of cables is positioned in a respective groove of the plurality of grooves so that the cables are embedded in the base layer.

14. The foot orthotic of claim 13, wherein:
the plurality of sensors has a maximum sensor thickness; and
the plurality of cavities has a second thickness that is greater than the maximum sensor thickness within a predetermined range so that the plurality of sensors can sense changes in pressure applied by the user's foot.

15. The foot orthotic of claim 8, further comprising:
a cover layer disposed adjacent to a first surface of the base layer so that at least a portion of the cover layer is in physical contact with the plurality of sensors; and
the cover layer is configured to be adjacent to a sole of a user's foot while using the foot orthotic.

16. The foot orthotic of claim 1, wherein:
the device frame includes one or more flexible members configured to hold the detachable electronic device in position; and
the one or more flexible members are configured to apply force against a portion of the detachable electronic device so that the device frame surrounds at least a portion of the detachable electronic device when attached.

17. The foot orthotic of claim 1, wherein:
the device frame includes a snap-in region configured to fasten the detachable electronic device in position when the detachable electronic device is inserted; and
the snap-in region is configured to allow a protruding member of the detachable electronic device to be fastened at the snap-in region so that the device frame surrounds at least a portion of the detachable electronic device when the detachable electronic device is inserted.

18. The foot orthotic of claim 1, wherein the composite core includes:
a first surface that is configured to be adjacent to a sole of a user while in use;
a second surface that is opposite the first surface; and
a perimeter, wherein at least a portion of the perimeter is curved towards the first surface so that the first surface includes concave contours and the second surface includes convex contours.

19. The foot orthotic of claim 18, wherein:
the convex contours of the second surface of the composite core have predefined radii of curvature so that the foot orthotic has a small volume along the perimeter of the composite core.

20. A portable biomechanical assessment system, the portable biomechanical assessment system comprising:
a detachable electronic device having one or more first connector pins;
a device frame configured to secure the detachable electronic device;
a transitional layer that includes a cut-out configured to accommodate the device frame and the detachable electronic device;
a base layer that at least partially surrounds the transitional layer; and
a printed circuit board disposed inside the cut-out of the transitional layer, wherein:
the base layer includes a cavity that is configured to accommodate the transitional layer;
the printed circuit board includes one or more second connector pins; and
the device frame is configured to secure the detachable electronic device in position so that electrical contact is maintained between the one or more first connector pins of the detachable electronic device and the one or more second connector pins of the printed circuit board while the detachable electronic device is attached in the device frame.
